SEQUENCE LISTING (56 sequences)

SEQ ID NO: 1 - protein, 862 aa, Homo sapiens
MRMCTPIRGLLMALAVMFGTAMAFAPIPRITWEHREVHLVQFHEPDIYNYSALLLSEDKDTLYIGAREAVFAVNALNISEKQHEVYWKVSEDKKAKCAEKGKSKQTECLNYIRVLQPLSATSLYVCGTNAFQPACDHLNLTSFKFLGKNEDGKGRCPFDPAHSYTSVMVDGELYSGTSYNFLGSEPIISRNSSHSPLRTEYAIPWLNEPSFVFADVIRKSPDSPDGEDDRVYFFFTEVSVEYEFVFRVLIPRIARVCKGDQGGLRTLQKKWTSFLKARLICSRPDSGLVFNVLRDVFVLRSPGLKVPVFYALFTPQLNNVGLSAVCAYNLSTAEEVFSHGKYMQSTTVEQSHTKWVRYNGPVPKPRPGACIDSEARAANYTSSLNLPDKTLQFVKDHPLMDDSVTPIDNRPRLIKKDVNYTQIVVDRTQALDGTVYDVMFVSTDRGALHKAISLEHAVHIIEETQLFQDFEPVQTLLLSSKKGNRFVYAGSNSGVVQAPLAFCGKHGTCEDCVLARDPYCAWSPPTATCVALHQTESPSRGLIQEMSGDASVCPDKSKGSYRQHFFKHGGTAELKCSQKSNLARVFWKFQNGVLKAESPKYGLMGRKNLLIFNLSEGDSGVYQCLSEERVKNKTVFQVVAKHVLEVKVVPKPVVAPTLSVVQTEGSRIATKVLVASTQGSSPPTPAVQATSSGAITLPPKPAPTGTSCEPKIVINTVPQLHSEKTMYLKSSDNRLLMSLFLFFFVLFLCLFFYNCYKGYLPRQCLKFRSALLIGKKKPKSDFCDREQSLKETLVEPGSFSQQNGEHPKPALDTGYETEQDTITSKVPTDREDSQRIDDLSARDKPFDVKCELKFADSDADGD

SEQ ID NO: 2 - protein, 861 aa, Murine sp.
MRMCAPVRGLFLALVVVLRTAVAFAPVPRLTWEHGEVGLVQFHKPGIFNYSALLMSEDKDTLYVGAREAVFAVNALNISEKQHEVYWKVSEDKKSKCAEKGKSKQTECLNYIRVLQPLSSTSLYVCGTNAFQPTCDHLNLTSFKFLGKSEDGKGRCPFDPAHSYTSVMVGGELYSGTSYNFLGSEPIISRNSSHSPLRTEYAIPWLNEPSFVFADVIQKSPDGPEGEDDKVYFFFTEVSVEYEFVFKLMIPRVARVCKGDQGGLRTLQKKWTSFLKARLICSKPDSGLVFNILQDVFVLRAPGLKEPVFYAVFTPQLNNVGLSAVCAYTLATVEAVFSRGKYMQSATVEQSHTKWVRYNGPVPTPRPGACIDSEARAANYTSSLNLPDKTLQFVKDHPLMDDSVTPIDNRPKLIKKDVNYTQIVVDRTQALDGTFYDVMFISTDRGALHKAVILTKEVHVIEETQLFRDSEPVLTLLLSSKKGRKFVYAGSNSGVVQAPLAFCEKHGSCEDCVLARDPYCAWSPAIKACVTLHQEEASSRGWIQDMSGDTSSCLDKSKESFNQHFFKHGGTAELKCFQKSNLARVVWKFQNGELKAASPKYGFVGRKHLLIFNLSDGDSGVYQCLSEERVRNKTVSQLLAKHVLEVKMVPRTPPSPTSEDAQTEGSKITSKMPVASTQGSSPPTPALWATSPRAATLPPKSSSGTSCEPKMVINTVPQLHSEKTVYLKSSDNRLLMSLLLFIFVLFLCLFSYNCYKGYLPGQCLKFRSALLLGKKTPKSDFSDLEQSVKETLVEPGSFSQQNGDHPKPALDTGYETEQDTITSKVPTDREDSQRIDELSARDKPFDVKCELKFADSDADGD

SEQ ID NO: 3 - DNA, 30 bp, Artificial
  Polynucleotide anti-CD100 VH CDR1
ggctacagcttcagcgactactacatgcac

SEQ ID NO: 4 - DNA, 51 bp, Artificial
  Polynucleotide anti-CD100 VH CDR2
cagattaatcctaccactggcggcgctagctacaaccagaagttcaagggc

SEQ ID NO: 5 - DNA, 27 bp, Artificial
  Polynucleotide anti-CD100 VH CDR3
tattactacggcagacacttcgatgtc

SEQ ID NO: 6 - protein, 10 aa, Artificial
  Polypeptide anti-CD100 VH CDR1
GYSFSDYYMH

SEQ ID NO: 7 - protein, 17 aa, Artificial
  Polypeptide anti-CD100 VH CDR2
QINPTTGGASYNQKFKG

SEQ ID NO: 8 - protein, 9 aa, Artificial
  Polypeptide anti-CD100 VH CDR3
YYYGRHFDV

SEQ ID NO: 9 - protein, 118 aa, Artificial
  Polypeptide anti-CD100 VH 2503
QVQLVQSGAEVKKPGSSVKVSCKASGYSFSDYYMHWVRQAPGQGLEWMGQINPTTGGASYNQKFKGKATITVDKSTSTAYMELSSLRSEDTAVYYCARYYYGRHFDVWGQGTTVTVSS

SEQ ID NO: 10 - protein, 118 aa, Artificial
  Polypeptide anti-CD100 VH 67
QVQLQQSGPELVKPGASVKISCKASGYSFSDYYMHWVKQSPENSLEWIGQINPTTGGASYNQKFKGKATLTVDKSSSTAYMQLKSLTSEESAVYYCTRYYYGRHFDVWGQGTTVTVSS

SEQ ID NO: 11 - DNA, 45 bp, Artificial
  Polynucleotide anti-CD100 VL CDR1
aaggccagccaaagcgtggattatgatggcgatagctatatgaac

SEQ ID NO: 12 - DNA, 21 bp, Artificial
  Polynucleotide anti-CD100 VL CDR2
gctgcatccaatctggaaagc

SEQ ID NO: 13 - DNA, 27 bp, Artificial
  Polynucleotide anti-CD100 VL CDR3
cagcaaagcaatgaggatccctacacc

SEQ ID NO: 14 - protein, 15 aa, Artificial
  Polypeptide anti-CD100 VL CDR1
KASQSVDYDGDSYMN

SEQ ID NO: 15 - protein, 7 aa, Artificial
  Polypeptide anti-CD100 VL CDR2
AASNLES

SEQ ID NO: 16 - protein, 9 aa, Artificial
  Polypeptide anti-CD100 VL CDR3
QQSNEDPYT

SEQ ID NO: 17 - protein, 111 aa, Artificial
  Polypeptide anti-CD100 VL 2503
DIVMTQSPDSLAVSLGERATINCKASQSVDYDGDSYMNWYQQKPGQPPKLLIYAASNLESGVPDRFSGSGSGTDFTLTISSLQAEDVAVYYCQQSNEDPYTFGQGTKLEIK

SEQ ID NO: 18 - protein, 111 aa, Artificial
  Polypeptide anti-CD100 VL 67
DIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 19 - DNA, 354 bp, Artificial
  Polynucleotide anti-CD100 VH 2503
caggtgcagctggtgcagagcggcgctgaggtgaagaagcctggcagcagcgtgaaggtctcctgcaaggctagcggctacagcttcagcgactactacatgcactgggtgagacaggcccctggccaaggcctggagtggatgggccagattaatcctaccactggcggcgctagctacaaccagaagttcaagggcaaggccaccattaccgtggacaaaagcaccagcacagcctacatggagctgagcagcctgagaagcgaggacaccgccgtgtattactgtgccagatattactacggcagacacttcgatgtctggggccaaggcaccacggtcaccgtctcttca

SEQ ID NO: 20 - DNA, 354 bp, Artificial
  Polynucleotide anti-CD100 VH 67
caggtccagctgcagcagtctggacctgagctggtgaagcctggggcttcagtgaagatatcctgcaaggcttctggttactcattcagtgactactacatgcactgggtgaagcaaagtcctgaaaatagtcttgagtggattggacagattaatcctaccactgggggtgctagctacaaccagaagttcaagggcaaggccacattaactgtagataaatcctccagcacagcctacatgcagctcaagagcctgacatctgaagagtctgcagtctattactgtacaagatattactacggtagacacttcgatgtctggggccaagggaccacggtcaccgtttcctca

SEQ ID NO: 21 - DNA, 333 bp, Artificial
  Polynucleotide anti-CD100 VL 2503
gacatcgtgatgacccagagcccagacagcctggctgtgagcctgggcgagagggccaccatcaactgcaaggccagccaaagcgtggattatgatggcgatagctatatgaactggtaccagcagaaaccaggccagcctcctaagctgctgatttacgctgcatccaatctggaaagcggcgtgcctgacagattcagcggcagcggcagcggcacagatttcactctgaccatcagcagcctgcaggctgaagatgtggcagtgtattactgtcagcaaagcaatgaggatccctacaccttcggccaagggaccaagctcgagatcaaa

SEQ ID NO: 22 - DNA, 333 bp, Artificial
  Polynucleotide anti-CD100 VL 67
gacattgtgatgacccagtctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctcgagatcaaa

SEQ ID NO: 23 - DNA, 2586 bp, Homo sapiens
atgaggatgtgcacccccattagggggctgctcatggcccttgcagtgatgtttgggacagcgatggcatttgcacccataccccggatcacctgggagcacagagaggtgcacctggtgcagtttcatgagccagacatctacaactactcagccttgctgctgagcgaggacaaggacaccttgtacataggtgcccgggaggcggtcttcgctgtgaacgcactcaacatctccgagaagcagcatgaggtgtattggaaggtctcagaagacaaaaaagcaaaatgtgcagaaaaggggaaatcaaaacagacagagtgcctcaactacatccgggtgctgcagccactcagcgccacttccctttacgtgtgtgggaccaacgcattccagccggcctgtgaccacctgaacttaacatcctttaagtttctggggaaaaatgaagatggcaaaggaagatgtccctttgacccagcacacagctacacatccgtcatggttgatggagaactttattcggggacgtcgtataattttttgggaagtgaacccatcatctcccgaaattcttcccacagtcctctgaggacagaatatgcaatcccttggctgaacgagcctagtttcgtgtttgctgacgtgatccgaaaaagcccagacagccccgacggcgaggatgacagggtctacttcttcttcacggaggtgtctgtggagtatgagtttgtgttcagggtgctgatcccacggatagcaagagtgtgcaagggggaccagggcggcctgaggaccttgcagaagaaatggacctccttcctgaaagcccgactcatctgctcccggccagacagcggcttggtcttcaatgtgctgcgggatgtcttcgtgctcaggtccccgggcctgaaggtgcctgtgttctatgcactcttcaccccacagctgaacaacgtggggctgtcggcagtgtgcgcctacaacctgtccacagccgaggaggtcttctcccacgggaagtacatgcagagcaccacagtggagcagtcccacaccaagtgggtgcgctataatggcccggtacccaagccgcggcctggagcgtgcatcgacagcgaggcacgggccgccaactacaccagctccttgaatttgccagacaagacgctgcagttcgttaaagaccaccctttgatggatgactcggtaaccccaatagacaacaggcccaggttaatcaagaaagatgtgaactacacccagatcgtggtggaccggacccaggccctggatgggactgtctatgatgtcatgtttgtcagcacagaccggggagctctgcacaaagccatcagcctcgagcacgctgttcacatcatcgaggagacccagctcttccaggactttgagccagtccagaccctgctgctgtcttcaaagaagggcaacaggtttgtctatgctggctctaactcgggcgtggtccaggccccgctggccttctgtgggaagcacggcacctgcgaggactgtgtgctggcgcgggacccctactgcgcctggagcccgcccacagcgacctgcgtggctctgcaccagaccgagagccccagcaggggtttgattcaggagatgagcggcgatgcttctgtgtgcccggataaaagtaaaggaagttaccggcagcattttttcaagcacggtggcacagcggaactgaaatgctcccaaaaatccaacctggcccgggtcttttggaagttccagaatggcgtgttgaaggccgagagccccaagtacggtcttatgggcagaaaaaacttgctcatcttcaacttgtcagaaggagacagtggggtgtaccagtgcctgtcagaggagagggttaagaacaaaacggtcttccaagtggtcgccaagcacgtcctggaagtgaaggtggttccaaagcccgtagtggcccccaccttgtcagttgttcagacagaaggtagtaggattgccaccaaagtgttggtggcatccacccaagggtcttctcccccaaccccagccgtgcaggccacctcctccggggccatcacccttcctcccaagcctgcgcccaccggcacatcctgcgaaccaaagatcgtcatcaacacggtcccccagctccactcggagaaaaccatgtatcttaagtccagcgacaaccgcctcctcatgtccctcttcctcttcttctttgttctcttcctctgcctctttttctacaactgctataagggatacctgcccagacagtgcttgaaattccgctcggccctactaattgggaagaagaagcccaagtcagatttctgtgaccgtgagcagagcctgaaggagacgttagtagagccagggagcttctcccagcagaatggggagcaccccaagccagccctggacaccggctatgagaccgagcaagacaccatcaccagcaaagtccccacggatagggaggactcacagaggatcgacgacctttctgccagggacaagccctttgacgtcaagtgtgagctgaagttcgctgactcagacgcagatggagac

SEQ ID NO: 24 - protein, 13 aa, Artificial
  Peptide epitope of proteolipid protein PLP (139-151)
HSLGKWLGHPDKF

SEQ ID NO: 25 - protein, 118 aa, Artificial
  Polypeptide anti-CD100 VH 76
QVQLQQSGAELAKPGASVKMSCKASGYTFTRYWMHWVKQRPGQGLEWIGYINPSTGYSDYNQKFKDKATLTADKSSSTAYMQLSSLTSEDSAVYYCARDPYGWTMDSWGQGTLVTVSS

SEQ ID NO: 26 - protein, 10 aa, Artificial
  Polypeptide anti-CD100 VH 76 CDR1
GYTFTRYWMH

SEQ ID NO: 27 - protein, 17 aa, Artificial
  Polypeptide anti-CD100 VH 76 CDR2
YINPSTGYSDYNQKFKD

SEQ ID NO: 28 - protein, 9 aa, Artificial
  Polypeptide anti-CD100 VH 76 CDR3
DPYGWTMDS

SEQ ID NO: 29 - protein, 107 aa, Artificial
  Polypeptide anti-CD100 VL 76
DIQMTQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLEIK

SEQ ID NO: 30 - protein, 11 aa, Artificial
  Polypeptide anti-CD100 VL 76 CDR1
HASQNINVWLS

SEQ ID NO: 31 - protein, 7 aa, Artificial
  Polypeptide anti-CD100 VL 76 CDR2
KASNLHT

SEQ ID NO: 32 - protein, 9 aa, Artificial
  Polypeptide anti-CD100 VL 76 CDR3
QQGQSYPYT

SEQ ID NO: 33 - DNA, 354 bp, Artificial
  Polynucleotide anti-CD100 VH 76
caggtccagctgcagcagtctggggctgaactggcaaaacctggggcctcagtgaagatgtcctgcaaggcttctggctacacctttactaggtactggatgcactgggtaaaacagaggcctggacagggtctggaatggattggatacattaatcctagcactggttattctgattacaatcagaagttcaaggacaaggccacattgactgcagacaaatcctccagcacagcctacatgcaactgagcagcctgacatctgaggactctgcagtctattactgtgcaagagacccctacggctggactatggactcctggggccaagggactctggtcaccgtctcctca

SEQ ID NO: 34 - DNA, 30 bp, Artificial
  Polynucleotide anti-CD100 VH 76 CDR1
ggctacacctttactaggtactggatgcac

SEQ ID NO: 35 - DNA, 51 bp, Artificial
  Polynucleotide anti-CD100 VH 76 CDR2
tacattaatcctagcactggttattctgattacaatcagaagttcaaggac

SEQ ID NO: 36 - DNA, 27 bp, Artificial
  Polynucleotide anti-CD100 VH 76 CDR3
gacccctacggctggactatggactcc

SEQ ID NO: 37 - DNA, 321 bp, Artificial
  Polynucleotide anti-CD100 VL 76
gacatccagatgacccagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatccgtacacgttcggaggggggaccaagctcgagatcaaa

SEQ ID NO: 38 - DNA, 33 bp, Artificial
  Polynucleotide anti-CD100 VL 76 CDR1
catgccagtcagaacattaatgtttggttaagc

SEQ ID NO: 39 - DNA, 21 bp, Artificial
  Polynucleotide anti-CD100 VL 76 CDR2
aaggcttccaacttgcacaca

SEQ ID NO: 40 - DNA, 27 bp, Artificial
  Polynucleotide anti-CD100 VL 76 CDR3
caacagggtcaaagttatccgtacacg

SEQ ID NO: 41 - DNA, 51 bp, Artificial
  Epitope 1
ctgaaggtgcctgtgttctatgcactcttcaccccacagctgaacaacgtg

SEQ ID NO: 42 - protein, 17 aa, Artificial
  Epitope 1
LKVPVFYALFTPQLNNV

SEQ ID NO: 43 - DNA, 45 bp, Artificial
  Epitope 2
aaatggacctccttcctgaaagcccgactcatctgctcccggcca

SEQ ID NO: 44 - protein, 15 aa, Artificial
  Epitope 2
KWTSFLKARLIASRP

SEQ ID NO: 45 - DNA, 42 bp, Artificial
  Epitope 3
gagtttgtgttcagggtgctgatcccacggatagcaagagtg

SEQ ID NO: 46 - protein, 14 aa, Artificial
  Epitope 3
EFVFRVLIPRIARV

SEQ ID NO: 47 - protein, 113 aa, Artificial sequence
  2282 VL domain
DIVMTQSPLSLPVSPGEPATINCKSSQSLFNSGNQKNYLAWYLQKPGQPPKLLIYGASTRESGVPDRFSGSGSGTDFTLTISSLQAEDVAVYYCQNDHTYPYTFGQGTKLEIK

SEQ ID NO: 48 - protein, 122 aa, Artificial sequence
  2282 VH domain
QVQLVQSGAEVKKPGSSVKVSCTASGYTFTDYYMDWVRQAPGQGLEWMGRVNPYHGYATYNQKFKGRVTITADKSTSTAYMELSSLRSEDTAVYYCAREENSYDGYYGMDYWGQGTLVTVSS

SEQ ID NO: 49 - protein, 113 aa, Artificial Sequence
  VX18 VH
EVQLVESGGGLVQPGGSLRLSCAASGFIFSDYWMVWVRQAPGKGLEYVAHMNQDGGARYYAESVRGRFTISRDNAKNSLYLQMNSLRAEDTAVYYCARDPWGYDSWGQGTLVT

SEQ ID NO: 50 - protein, 5 aa, Artificial Sequence
  VX18 VH CDR1
DYWMV

SEQ ID NO: 51 - protein, 11 aa, Artificial Sequence
  VX18 VH CDR2
HMNQDGGARYY

SEQ ID NO: 52 - protein, 5 aa, Artificial Sequence
  VX18 VH CDR3
DPWGY

SEQ ID NO: 53 - protein, 104 aa, Artificial Sequence
  VX18 VL
SYELTQPPSVSVSPGQTASITCSGDKLGDKYAVWYQQKPGQSPVLVIYQDSKRPSGIPERFSGSNSGNTATLTISGTQAMDEADYYCQAWEQEAAWVFGGGTKL

SEQ ID NO: 54 - protein, 11 aa, Artificial Sequence
  VX18 VL CDR1
SGDKLGDKYAV

SEQ ID NO: 55 - protein, 7 aa, Artificial Sequence
  VX18 VL CDR2
QDSKRPS

SEQ ID NO: 56 - protein, 10 aa, Artificial Sequence
  VX18 VL CDR3
QAWEQEAAWV

(19) United States
(12) Patent Application Publication
(10) Pub. No.: US 2021/0032322 A1
(43) Pub. Date: Feb. 4, 2021

(54) COMBINED INHIBITION OF SEMAPHORIN-4D AND TGF-BETA AND COMPOSITIONS THEREFOR

(71) Applicant: Vaccinex, Inc., Rochester, NY (US)

(72) Inventors: Elizabeth E. Evans, Bloomfield, NY (US); Holm Bussler, Rochester, NY (US)

(73) Assignee: Vaccinex, Inc., Rochester, NY (US)

(21) Appl. No.: 16/943,107

(22) Filed: Jul. 30, 2020

Related U.S. Application Data
(60) Provisional application No. 62/881,751, filed on Aug. 1, 2019.

(51) Int. Cl. C07K 16/22; A61P 35/00; C07K 16/28

(52) U.S. Cl. CPC C07K 16/22; A61P 35/00; C07K 16/2827; C07K 2317/76; C07K 16/2818; C07K 2317/565; C07K 16/2851

(57) ABSTRACT

Provided herein are methods for inhibiting, delaying, or reducing tumor growth and metastases of plexin-B1, plexin-B2, and/or CD72-expressing cancer cells in a subject, comprising administering to the subject an effective amount of an isolated binding molecule which specifically binds to semaphorin-4D (SEMA4D) in combination with an effective amount of antibody or antigen-binding fragment thereof that inhibits TGFβ, and, optionally, at least one other immune modulating therapy.

### CROSS-REFERENCE TO RELATED APPLICATION

This is a non-provisional of pending U.S. provisional application Ser. No. 62/881,751, filed Aug. 1, 2019, the entirety of which application is incorporated by reference herein.

### REFERENCE TO A SEQUENCE LISTING SUBMITTED VIA EFS-WEB

The content of the ASCII text file of the sequence listing named “36P1_ST25” which is 37 kb in size was created on Aug. 1, 2019, and electronically submitted via EFS-Web herewith the application is incorporated herein by reference in its entirety.

### BACKGROUND

Semaphorin 4D (SEMA4D), also known as CD100, is a transmembrane protein (e.g., SEQ ID NO: 1 (human); SEQ ID NO: 2 (murine)) that belongs to the semaphorin gene family. SEMA4D is expressed on the cell surface as a homodimer, but upon cell activation SEMA4D can be released from the cell surface via proteolytic cleavage to generate sSEMA4D, a soluble form of the protein, which is also biologically active. See Suzuki et al., Nature Rev. Immunol 3: 159-167 (2003); Kikutani et al., Nature Immunol 9: 17-23 (2008).

SEMA4D is expressed at elevated levels in lymphoid organs, including the spleen, thymus, and lymph nodes, and in non-lymphoid organs, such as the brain, heart, and kidney. In lymphoid organs, SEMA4D is abundantly expressed on resting T cells but only weakly expressed on resting B cells and antigen-presenting cells (APCs), such as dendritic cells (DCs). Its expression, however, is upregulated in these cells following activation by various immunological stimuli. The release of soluble SEMA4D from immune cells is also increased by cell activation. SEMA4D has been implicated in the development of certain cancers (Ch'ng et al., Cancer 110: 164-72 (2007); Campos et al., Oncology Letters, 5: 1527-35 (2013); Kato et al., Cancer Sci 102: 2029-37 (2011)) and several reports suggest that one mechanism of this influence is the role of SEMA4D in promoting tumor angiogenesis (Conrotto et al., Blood 105: 4321-4329 (2005). Basile et al., J Biol Chem 282: 34888-34895 (2007); Sierra et al., J Exp Med 205: 1673 (2008); Zhou et al., Angiogenesis 15: 391-407 (2012)). Tumor growth and metastasis involve a complex process of cross talk amongst the tumor cells, stroma and immune infiltrate, as well as the endothelial cells and vasculature. SEMA4D is over-expressed in a wide array of tumor types and is also produced by inflammatory cells recruited to the tumor microenvironment, the question of what role SEMA4D can play in migration, survival, differentiation, and organization of the different cell types that constitute the tumor stroma remains to be addressed.

### SUMMARY

This application addresses the need for safe and effective cancer treatments that inhibit, reduce, suppress, prevent, slow or delay the progression of, shrink, or directly attack tumor cells, or that can act in combination with other immune modulating therapies to enhance their therapeutic benefit. In particular, SEMA4D was shown to play a role in the infiltration, maturation and organization of immune cells and macrophage that either promote or inhibit tumor growth, which can contribute to development of effective methods for reducing tumor growth and metastases in a subject with cancer.

Certain aspects of the application are directed to a method for inhibiting, delaying, or reducing tumor growth or metastases or both tumor growth and metastases in a subject with cancer comprising administering to the subject an effective amount of an isolated binding molecule which specifically binds to semaphorin-4D (SEMA4D) and an effective amount of an agent that inhibits TGFβ. In some embodiments, the agent that inhibits TGFβ comprises an effective amount of an anti-TGFβ antibody or antigen-binding fragment thereof. In some embodiments, the administration of an anti-TGFβ antibody or antigen-binding fragment thereof is administered in combination with another immune modulating therapy.

In some embodiments, the binding molecule inhibits SEMA4D interaction with its receptor (e.g., Plexin-B1, Plexin-B2, and/or CD72). In some embodiments, the binding molecule inhibits SEMA4D-mediated Plexin-B1, Plexin-B2, and/or CD72 signal transduction. In some embodiments, the inhibition, delay, or reduction of metastases occurs independently of primary tumor growth inhibition, delay, or reduction. In some embodiments, the cancer is selected from the group consisting of carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, and a combination thereof. In some embodiments, the subject has elevated levels of either B cells, T cells or both B cells and T cells when compared to other cancer subjects.

In some embodiments, the isolated binding molecule specifically binds to the same SEMA4D epitope as a reference monoclonal antibody selected from the group consisting of VX15/2503 and 67. In some embodiments, the isolated binding molecule comprises an antibody or antigen-binding fragment thereof. In some embodiments, the antibody or antigen-binding fragment thereof comprises the six complementarity determining regions (CDRs) of monoclonal antibody VX15/2503 or 67.

In some embodiments, the immune modulating therapy comprises an anti-TGFβ antibody or antigen-binding fragment thereof, optionally in combination with an additional immune modulating therapy. In some embodiments, the additional immune modulating therapy is selected from the group consisting of a cancer vaccine, an immunostimulatory agent, adoptive T cell or antibody therapy, immune checkpoint blockade and a combination thereof. In some embodiments, the isolated binding molecule and the immune modulating agent or immune modulating therapy are administered separately or concurrently.

In some embodiments, the isolated binding molecule specifically binds to the same SEMA4D epitope as a reference monoclonal antibody VX15/2503 or 67. In some embodiments, the isolated binding molecule competitively inhibits a reference monoclonal antibody VX15/2503 or 67 from specifically binding to SEMA4D. In some embodiments, the isolated binding molecule comprises an antibody or antigen-binding fragment thereof. In some embodiments, the antibody or antigen-binding fragment thereof comprises a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively. In some embodiments, the VH and VL comprise, respectively, SEQ ID NO: 9 and SEQ ID NO: 17 or SEQ ID NO: 10 and SEQ ID NO: 18.

In some embodiments, the anti-TGFβ antibody or antigen-binding fragment thereof is a bispecific antibody. In some embodiments, the bispecific antibody provides a combination of anti-TGFβ and an additional immune modulating therapy. In some embodiments, the additional immune modulating therapy comprises an immune checkpoint blockade inhibitor. In some embodiments, the immune checkpoint blockade inhibitor is an anti-PD-1 antibody. In some embodiments, the immune checkpoint blockade inhibitor is an anti-PD-L1 antibody. In some embodiments, the immune checkpoint blockade inhibitor is an anti-CTLA-4 antibody. In some embodiments, the immune checkpoint blockade inhibitor is an anti-LAG3 antibody. In some embodiments, the isolated binding molecule and the immune modulating therapy are administered separately or concurrently. In some embodiments, administration of the combination of the isolated binding molecule and the immune modulating therapy results in enhanced therapeutic efficacy relative to administration of the isolated binding molecule or the immune modulating therapy alone. In some embodiments, the subject has an elevated level of B cells, T cells or both B cells and T cells when compared to other cancer subjects. In some embodiments, the level of B cells and/or T cells per microliter of blood in the subject is about 1.5, 2, 2.5, 3, 3.5, 4, 4.5, or 5 times the mean number of B cells and/or T cells in circulation in other cancer patients. In some embodiments, the level of B cells and/or T cells per microliter of blood in the subject ranges from about 147 to about 588 and from about 1173 to about 3910, respectively, e.g., when compared to other cancer patients. In some embodiments, the subject has B cell and/or T cell levels that fall within or above the range of B cells and/or T cells of healthy, non-cancer patients. In some embodiments, the B cell and/or T cell levels per microliter of blood in the subject range from about 225 to about 275 or more and from about 1350 to about 1650 or more, respectively, e.g., when compared to healthy, non-cancer patients.

In some embodiments, methods for treating a subject having cancer with immunotherapy are provided that comprise: (a) determining the number of B cells and/or T cells in a subject with cancer; and (b) administering to the subject an effective amount of an isolated binding molecule which specifically binds to semaphorin-4D (SEMA4D) and an effective amount of at least one other immune modulating therapy if the number of B cells and/or T cells in the subject exceeds a predetermined threshold level. In some embodiments, the predetermined threshold levels of B cells and/or T cells per microliter of blood in the subject is about 1.5, 2, 2.5, 3, 3.5, 4, 4.5, or 5 times the mean number of B cells and/or T cells in circulation in other cancer patients. In some embodiments, the predetermined threshold levels of B cells and/or T cells per microliter of blood in the subject range from about 147 to about 588 and from about 1173 to about 3910, respectively, e.g., when compared to other cancer patients. In some embodiments, the predetermined threshold levels of B cells and/or T cells per microliter of blood in the subject fall within or above the range of B cells and/or T cells of healthy, non-cancer patients. In some embodiments, the predetermined threshold levels of B cells and/or T cells per microliter of blood in the subject range from about 225 to about 275 or more and from about 1350 to about 1650, or more, respectively, e.g., when compared to healthy, non-cancer patients.

In some embodiments, methods of treating a subject having cancer with immunotherapy are provided that comprise: administering a combination of an effective amount of an isolated binding molecule that specifically binds to semaphorin-4D (SEMA4D) and an effective amount of an anti-TGFβ antibody or antigen-binding fragment thereof to a subject with cancer, wherein administration of the combination results in enhanced therapeutic efficacy relative to administration of the isolated binding molecule or the an anti-TGFβ antibody or antigen-binding fragment thereof alone. In some embodiments, the anti-TGFβ antibody or antigen-binding fragment thereof, is administered alone or in combination with an additional immune modulating therapy. In some embodiments, the additional immune modulating therapy is administration of an immune checkpoint blockade inhibitor. The additional immune modulatory therapy can be administered as a separate molecule or composition, or as part of the same molecule or composition. In some embodiments, the immune modulating therapy comprises an anti-TGFβ antibody or antigen-binding fragment thereof that is bispecific for TGFβ and another antigen. In some embodiments, the immune modulating therapy comprises an immune checkpoint blockade inhibitor. In some embodiments, the immune checkpoint blockade inhibitor is an anti-PD-1 antibody, administered alone or as a combination with the anti-TGFβ antibody or antigen-binding fragment thereof. In some embodiments, the isolated binding molecule and the immune modulating therapy are administered separately or concurrently.

### DESCRIPTION OF THE DRAWINGS

FIG. 1: Increased levels of pro-inflammatory cytokines IFNg and TNFa observed in the TIL of mice treated with anti-SEMA4D antibody. TIL from Colon26 tumor-bearing mice treated with anti-SEMA4D had higher levels of CXCL9, a chemokine that recruits CD8+ T cells into the tumor tissue. In these same mice, there was a reduction in immunosuppressive chemokines including CCL2, CXCL1 and CXCL5, which recruit and polarize immunosuppressive cells such as myeloid derived suppressor cells, M2 tumor associated macrophage, and T regulatory cells. Statistical significance was determined with Mann-Whitney non-parametric t test.

FIGS. 2A-2D: Measurement of tumor volume and survival in a colon cancer model. FIG. 2A shows measurement of mean tumor volume over time in C57B1/6J mice after subcutaneous implantation with MC38 tumor cells. Mice were treated with either control (MAB2B8.1E7, mouse IgG1, weekly for 2 weeks), anti-SEMA4D/MAb 67-2 (10 mg/kg, IP, weekly for 2 weeks), Anti-TGFb (MAB 1D11.16.8, BioXCell; 10 mg/kg, 3×/week for 3 weeks, starting when tumor volume reaches about 130 mm³ (10 days post tumor injection)), or both anti-TGFb and anti-SEMA4D. FIG. 2B shows percent survival over the same time period. FIG. 2C shows measurement of mean tumor volume over time in BALB/cJ (Jackson Labs) mice after subcutaneous implantation with Colon26 cells. Mice were treated with either control (MAB2B8.1E7, mouse IgG1, weekly for 3 weeks), anti-SEMA4D/MAb 67-2 (10 mg/kg, IP, weekly for 3 weeks), Anti-TGFb (MAB 1D11.16.8, BioXCell; 10 mg/kg, 3×/week for 3 weeks, starting when tumor volume reaches about 130 mm³ (10 days post tumor injection)), or both anti-TGFb and anti-SEMA4D. FIG. 2D shows percent survival over the same time period.

FIGS. 3A-3B: Measurement of tumor volume in mice implanted with syngeneic Colon26 tumor cells. FIG. 3A shows measurement of Colon26 tumor volume in Balb/c mice treated with either control Mouse IgG1/2B8 or anti-SEMA4D/MAb 67-2 (50 mg/kg, IP, weekly) together with either control rat Ig or rat anti-PD1/MAbRMP1-14 (100 μg, twice per week, for 2 weeks starting at 3 days post tumor inoculation). FIG. 3B shows survival time of Balb/c mice treated with either control Mouse IgG1/2B8 or anti-SEMA4D/MAb 67-2 together with either control rat Ig or rat anti-PD1/MAbRMP1-14.

FIG. 4A-4B: Measurement of tumor volume in mice implanted subcutaneously with 500,000 Colon26 tumor cells. FIG. 4A shows measurement of Colon26 tumor volume in Balb/c mice treated with either control Mouse IgG1/2B8 (10 mg/kg, weekly×2) or anti-SEMA4D/MAb 67 (10 mg/kg, weekly×2) and/or anti-PD-L1/MAb 10F.9G2 (10 mg/kg, twice weekly×4). FIG. 4B shows survival time. *p<0.05; **p<0.01; CR=complete tumor regression.

### DETAILED DESCRIPTION

Provided herein are methods of treating or inhibiting cancer in subjects which comprise administering to the subjects one or more anti-SEMA4D antibodies or fragments thereof in combination with one or more anti-TGFβ antibodies or fragments thereof.

### Definitions

As used herein, the terms “cancer” and “cancerous” refer to or describe the physiological condition in mammals in which a population of cells is characterized by unregulated cell growth. Examples of cancer include carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, gastric, pancreatic cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, sarcoma, kidney cancer, liver cancer, prostate cancer, vulval cancer, thyroid cancer, hepatic carcinoma and various types of head and neck cancers.

In some embodiments, the cancer is a metastatic cancer, such as metastatic sarcomas, breast carcinomas, ovarian cancer, head and neck cancer, and pancreatic cancer. In some embodiments, the cancer cells express Plexin-B1, Plexin-B2, and/or CD72 receptors for SEMA4D. In some embodiments, the cancer to be treated is a tumor. As used herein, “tumor” and “neoplasm” are used interchangeably to refer to any mass of tissue that result from abnormal cell growth or proliferation, either benign (noncancerous) or malignant (cancerous) including pre-cancerous lesions. In some embodiments, the tumors to be treated express Plexin-B1 and/or Plexin-B2 and/or CD72, and can express SEMA4D and activated Met.

The term “immune modulating therapy” or “immunotherapy” refers to treatment that impacts a disease or disorder in a subject by inducing and/or enhancing an immune response in that subject. Immune modulating therapies include cancer vaccines, immunostimulatory agents, adoptive T cell or antibody therapy, and immune checkpoint blockade (Lizée et al. (2013) Annu Rev Med 64: 71-90).

The term “immune modulating agent” refers to the active agents of immunotherapy. Immune modulating agents include a diverse array of recombinant, synthetic and natural, preparation. Examples of immune modulating agents include interleukins such as IL-2, IL-7, IL-12; cytokines such as granulocyte colony-stimulating factor (G-CSF), interferons; various chemokines such as CXCL13, CCL26, CXCL7; antagonists of immune checkpoint blockades such as anti-CTLA-4, anti-PD1 or anti-PD-L1 (ligand of PD-1), anti-LAG3, anti-B7-H3, anti-TGFβ, synthetic cytosine phosphate-guanosine (CpG) oligodeoxynucleotides, glucans; and modulators of regulatory T cells (Tregs) such as cyclophosphamide.

The term “therapeutically effective amount” refers to an amount of an antibody, polypeptide, polynucleotide, small organic molecule, or other drug effective to “treat” a disease or disorder in a subject or mammal. In the case of cancer, the therapeutically effective amount of the drug can reduce the number of cancer cells; retard or stop cancer cell division, reduce or retard an increase in tumor size; inhibit, e.g., suppress, retard, prevent, stop, delay, or reverse cancer cell infiltration into peripheral organs including, for example, the spread of cancer into soft tissue and bone; inhibit, e.g., suppress, retard, prevent, shrink, stop, delay, or reverse tumor metastasis; inhibit, e.g., suppress, retard, prevent, stop, delay, or reverse tumor growth; relieve to some extent one or more of the symptoms associated with the cancer, reduce morbidity and mortality; improve quality of life; or a combination of such effects. To the extent the drug prevents growth and/or kills existing cancer cells, it can be referred to as cytostatic and/or cytotoxic.

Terms such as “treating” or “treatment” or “to treat” or “alleviating” or “to alleviate” refer to both 1) therapeutic measures that cure, slow down, lessen symptoms of, reverse, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. A subject is successfully “treated” according to the methods of the present disclosure if the patient shows one or more of the following: a reduction in the number of or complete absence of cancer cells; a reduction in the tumor size; or retardation or reversal of tumor growth, inhibition, e.g., suppression, prevention, retardation, shrinkage, delay, or reversal of metastases, e.g., of cancer cell infiltration into peripheral organs including, for example, the spread of cancer into soft tissue and bone; inhibition of, e.g., suppression of, retardation of, prevention of, shrinkage of, reversal of, delay of, or an absence of tumor metastases; inhibition of, e.g., suppression of, retardation of, prevention of, shrinkage of, reversal of, delay of, or an absence of tumor growth; relief of one or more symptoms associated with the specific cancer; reduced morbidity and mortality; improvement in quality of life; or some combination of effects. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, a “binding molecule” refers to a molecule that specifically binds the referenced “target.” For example, an anti-SEMA4D binding molecule, specifically binds SEMA4D, e.g., a transmembrane SEMA4D polypeptide of about 150 kDa or a soluble SEMA4D polypeptide of about 120 kDa (commonly referred to as sSEMA4D). In some embodiments, a binding molecule is an antibody or an antigen binding fragment thereof. In some embodiments, a binding molecule comprises at least one heavy or light chain Complementarity Determining Region (CDR) of an antibody that binds or was raised against the referenced target. In some embodiments, a binding molecule comprises two, three, four, five or six CDRs from one or more antibodies that bind or were raised against the referenced target.

Unless specifically referring to full-sized antibodies such as naturally occurring antibodies, the term “anti-SEMA4D antibody” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules. Also included in SEMA4D binding molecules are other biologics or small molecules that bind and inhibit the activity of SEMA4D or of its Plexin-B1, Plexin-B2, and/or CD72 receptor.

As used herein, “human” or “fully human” antibodies include antibodies having the amino acid sequence of a human immunoglobulin and include antibodies isolated from human immunoglobulin libraries or from animals transgenic for one or more human immunoglobulins, as described infra and, for example, in U.S. Pat. No. 5,939,598 by Kucherlapati et al., “Human” or “fully human” antibodies also include antibodies comprising at least the variable domain of a heavy chain, or at least the variable domains of a heavy chain and a light chain, where the variable domain(s) have the amino acid sequence of human immunoglobulin variable domain(s).

“Human” or “fully human” antibodies also include “human” or “fully human” antibodies, as described above, that comprise, consist essentially of, or consist of, variants (including derivatives) of antibody molecules (e.g., the VH regions and/or VL regions) described herein, which antibodies or fragments thereof immunospecifically bind to a SEMA4D polypeptide or fragment or variant thereof. Standard techniques known to those of skill in the art can be used to introduce mutations in the nucleotide sequence encoding a human anti-SEMA4D antibody, including, but not limited to, site-directed mutagenesis and PCR-mediated mutagenesis which result in amino acid substitutions. In some embodiments, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3.

In some embodiments, the amino acid substitutions are conservative amino acid substitution, discussed further below. Alternatively, mutations can be introduced randomly along all or part of the coding sequence, such as by saturation mutagenesis, and the resultant mutants can be screened for biological activity to identify mutants that retain activity (e.g., the ability to bind a SEMA4D polypeptide, e.g., human, murine, or both human and murine SEMA4D). Such variants (or derivatives thereof) of “human” or “fully human” antibodies can also be referred to as human or fully human antibodies that are “optimized” or “optimized for antigen binding” and include antibodies that have improved affinity to antigen.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., (1988) Antibodies: A Laboratory Manual (2nd ed.; Cold Spring Harbor Laboratory Press).

As used herein, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL or VK) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs) within these variable domains, of an antibody combine to form the variable region that defines a three-dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule can consist of heavy chains only, with no light chains. See, e.g., Hamers-Casterman et al., Nature 363: 446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three-dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable domain by one of ordinary skill in the art, since they have been precisely defined (see below).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., (1983) U.S. Dept. of Health and Human Services, “Sequences of Proteins of Immunological Interest” and by Chothia and Lesk, J Mol Biol 196: 901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein.

Antibodies or antigen-binding fragments, variants, or derivatives thereof include polyclonal, monoclonal, multispecific, bispecific, human, humanized, primatized, or chimeric antibodies, single-chain antibodies, epitope-binding fragments, e.g., Fab, Fab′ and F(ab′)2, Fd, Fvs, single-chain Fvs (scFv), disulfide-linked Fvs (sdFv), fragments comprising either a VL or VH domain, fragments produced by a Fab expression library, and anti-idiotypic (anti-Id) antibodies (including, e.g., anti-Id antibodies to anti-SEMA4D antibodies disclosed herein). ScFv molecules are known in the art and are described, e.g., in U.S. Pat. No. 5,892,019. Immunoglobulin or antibody molecules can be of any type (e.g., IgG, IgE, IgM, IgD, IgA, and IgY), class (e.g., IgG1, IgG2, IgG3, IgG4, IgA1, and IgA2, etc.), or subclass of immunoglobulin molecule.

As used herein, the term “heavy chain portion” includes amino acid sequences derived from an immunoglobulin heavy chain. In some embodiments, a polypeptide comprising a heavy chain portion comprises at least one of: a VH domain, a CH1 domain, a hinge (e.g., upper, middle, and/or lower hinge region) domain, a CH2 domain, a CH3 domain, or a variant or fragment thereof. For example, a binding polypeptide for use in the disclosure can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In some embodiments, a polypeptide comprises a polypeptide chain comprising a CH3 domain. Further, a binding polypeptide for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

In some antibodies, or antigen-binding fragments, variants, or derivatives thereof, the heavy chain portions of one polypeptide chain of a multimer are identical to those on a second polypeptide chain of the multimer. Alternatively, in some embodiments, the heavy chain portion-containing monomers are not identical. For example, each monomer can comprise a different target binding site, forming, for example, a bispecific antibody. A bispecific antibody is an artificial protein that is composed of fragments of two different monoclonal antibodies and is consequently able to bind different antigenic epitopes. Variations on the bispecific antibody format are contemplated within the scope of the present disclosure. Bispecific antibodies can be generated using techniques that are known in the art. See, e.g., Ghayur et al., Expert Review of Clinical Pharmacology 3.4 (July 2010): p491; Lu et al., J Biological Chemistry 280(20): 19665-19672 (2005); Marvin et al., Acta Pharmacologic Sinica 26(6): 649-658 (2005); and Milstein et al., Nature 1983; 305: 537-40; 30 Brennan M et al., Science 1985; 229: 81-3; Thakur et al., Curr Opin Mol Ther 2010 Jun; 12(3): 340-9; and US 20070004909.

The heavy chain portions of a binding molecule for use in the methods disclosed herein can be derived from different immunoglobulin molecules. For example, a heavy chain portion of a polypeptide can comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain, e.g., a kappa or lambda light chain. In some embodiments, the light chain portion comprises at least one of a VL or CL domain.

Antibodies, antigen-binding fragments, variants, and derivatives thereof as disclosed herein may be described or specified in terms of their target antigen or epitope thereof that they recognize or specifically bind. The portion of a target polypeptide that specifically interacts with the antigen binding domain of an antibody is an “epitope,” or an “antigenic determinant.” A target polypeptide can comprise a single epitope or multiple epitopes depending on the size, conformation, and type of antigen. Epitopes can comprise linear amino acid residues (i.e., residues within the epitope that are arranged sequentially one after another in a linear fashion), nonlinear amino acid residues (referred to herein as “nonlinear epitopes” or “conformational epitopes”; these epitopes are not arranged sequentially), or both linear and nonlinear amino acid residues. Furthermore, it should be noted that an “epitope” on a target polypeptide can be or can include non-polypeptide elements, e.g., an epitope can include a carbohydrate side chain.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” can be deemed to have a higher specificity or affinity for a given epitope than antibody “B,” or antibody “A” can be said to bind to epitope “C” with a higher specificity or affinity than it has for related epitope “D.”

By “preferentially binds,” it is meant that the antibody specifically binds to an epitope more readily than it would bind to a related, similar, homologous, or analogous epitope. Thus, an antibody that “preferentially binds” to a given epitope would more likely bind to that epitope than to a related epitope, even though such an antibody can cross-react with the related epitope.

As used herein, the term “affinity” refers to a measure of the strength of the binding of an individual epitope with the CDR of an immunoglobulin molecule. See, e.g., Harlow et al., (1988) Antibodies: A Laboratory Manual (Cold Spring Harbor Laboratory Press, 2nd ed.) pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Anti-SEMA4D binding molecules, e.g., antibodies or antigen-binding fragments, variants or derivatives thereof can also be described or specified in terms of their binding affinity to a polypeptide of the disclosure, e.g., SEMA4D, e.g., human, murine, or both human and murine SEMA4D. In some embodiments, binding affinities include those with a dissociation constant or Kd less than 5×10⁻² M, 10⁻² M, 5×10⁻³ M, 10⁻³M, 5×10⁻⁴ M, 10⁻⁴ M, 5×10⁻⁵M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In some embodiments, the anti-SEMA4D binding molecule, e.g., an antibody or antigen binding fragment thereof, binds human SEMA4D with a Kd of about 5×10⁻⁹ to about 6×10⁻⁹. In some embodiments, the anti-SEMA4D binding molecule, e.g., an antibody or antigen binding fragment thereof, binds murine SEMA4D with a Kd of about 1×10⁻⁹ to about 2×10^{×9}.

As used herein, the term “chimeric antibody” will be held to mean any antibody wherein the immunoreactive region or site is obtained or derived from a first species and the constant region (which can be intact, partial or modified) is obtained from a second species. In some embodiments, the target binding region or site will be from a non-human source (e.g., mouse or primate) and the constant region is human.

As used herein, the term “engineered antibody” refers to an antibody in which the variable domain in either the heavy or light chain or both is altered by at least partial replacement of one or more CDRs from an antibody of known specificity and, if necessary, by partial framework region replacement and sequence changing. Although the CDRs can be derived from an antibody of the same class or even subclass as the antibody from which the framework regions are derived, it is envisaged that the CDRs will be derived from an antibody of different class or from an antibody from a different species. An engineered antibody in which one or more “donor” CDRs from a non-human antibody of known specificity is grafted into a human heavy or light chain framework region is referred to herein as a “humanized antibody.” In some embodiments, it is not necessary to replace all of the CDRs with the complete CDRs from the donor variable domain to transfer the antigen binding capacity of one variable domain to another. Rather, only those residues that are necessary to maintain the activity of the binding site against the targeted antigen can be transferred.

It is further recognized that the framework regions within the variable domain in a heavy or light chain, or both, of a humanized antibody can comprise solely residues of human origin, in which case these framework regions of the humanized antibody are referred to as “fully human framework regions” (for example, MAb VX15/2503, disclosed in U.S. Pat. No. 8,496,938, as MAb 2503, incorporated herein by reference in its entirety). Alternatively, one or more residues of the framework region(s) of the donor variable domain can be engineered within the corresponding position of the human framework region(s) of a variable domain in a heavy or light chain, or both, of a humanized antibody if necessary to maintain proper binding or to enhance binding to the SEMA4D antigen. A human framework region that has been engineered in this manner would thus comprise a mixture of human and donor framework residues, and is referred to herein as a “partially human framework region.”

For example, humanization of an anti-SEMA4D antibody can be essentially performed following the method of Winter and co-workers (Jones et al., Nature 321: 522-525 (1986); Riechmann et al., Nature 332: 323-327 (1988); Verhoeyen et al., Science 239: 1534-1536 (1988)), by substituting rodent or mutant rodent CDRs or CDR sequences for the corresponding sequences of a human anti-SEMA4D antibody. See also U.S. Pat. Nos. 5,225,539; 5,585,089; 5,693,761; 5,693,762; and 5,859,205; which are herein incorporated by reference in their entirety. The resulting humanized anti-SEMA4D antibody would comprise at least one rodent or mutant rodent CDR within the fully human framework regions of the variable domain of the heavy and/or light chain of the humanized antibody. In some instances, residues within the framework regions of one or more variable domains of the humanized anti-SEMA4D antibody are replaced by corresponding non-human (for example, rodent) residues (see, for example, U.S. Pat. Nos. 5,585,089; 5,693,761; 5,693,762; and 6,180,370), in which case the resulting humanized anti-SEMA4D antibody would comprise partially human framework regions within the variable domain of the heavy and/or light chain. Similar methods can be used for humanization of an anti-VEGF antibody.

Furthermore, humanized antibodies can comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the CDRs correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331: 522-525 (1986); Riechmann et al., Nature 332: 323-329 (1988); and Presta, Curr Op Struct Biol 2: 593-596 (1992); herein incorporated by reference. Accordingly, such “humanized” antibodies can include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; and 5,859,205. See also U.S. Pat. No. 6,180,370 and International Publication WO 01/27160, where humanized antibodies and techniques for producing humanized antibodies having improved affinity for a predetermined antigen are disclosed.

### SEMA4D, SEMA4D Epitopes, and Anti-SEMA4D Antibodies

As used herein, the terms “semaphorin-4D”, “SEMA4D”, and “SEMA4D polypeptide” are used interchangeably, as are “SEMA4D” and “Sema4D.” In some embodiments, SEMA4D is expressed on the surface of or secreted by a cell. In some embodiments, SEMA4D is membrane bound. In some embodiments, SEMA4D is soluble, e.g., sSEMA4D. In some embodiments, SEMA4D can include a full-sized SEMA4D or a fragment thereof, or a SEMA4D variant polypeptide, wherein the fragment of SEMA4D or SEMA4D variant polypeptide retains some or all functional properties of the full-sized SEMA4D. Use of anti-SEMA4D for the treatment of cancer has been described in, for example, Evans EE, et al., Antibody Blockade of Semaphorin 4D Promotes Immune Infiltration into Tumor and Enhances Response to Other Immunomodulatory Therapies. Cancer Immunol Res. 2015 Jun; 3(6):689-701, and also in, for example, U.S. Pat. Nos. 9,243,068 and 9,828,435.

The full-sized human SEMA4D protein is a homodimeric transmembrane protein consisting of two polypeptide chains of 150 kDa. SEMA4D belongs to the semaphorin family of cell surface receptors and is also referred to as CD100. Both human and mouse SEMA4D/Sema4D are proteolytically cleaved from their transmembrane form to generate 120-kDa soluble forms, giving rise to two Sema4D isoforms (Kumanogoh et al., J Cell Science 116(7): 3464 (2003)). Semaphorins consist of soluble and membrane-bound proteins that were originally defined as axonal-guidance factors which play an important role in establishing precise connections between neurons and their appropriate target. Structurally considered a class IV semaphorin, SEMA4D consists of an amino-terminal signal sequence followed by a characteristic ‘Sema’ domain, which contains 17 conserved cysteine residues, an Ig-like domain, a lysine-rich stretch, a hydrophobic transmembrane region, and a cytoplasmic tail.

Anti-SEMA4D antibodies are known in the art. See, for example, US 20080219971, US 20100285036, US 20060233793, WO 93/14125, WO 2008/100995, WO 2010/129917, WO 2018/204895, and Herold et al., Int Immunol 7(1): 1-8 (1995), each of which are herein incorporated by reference in their entirety.

The disclosure generally relates to a method of inhibiting, delaying, or reducing tumor growth or metastases in a subject, e.g., a human cancer patient, comprising administration of an antibody which specifically binds to SEMA4D, or an antigen-binding fragment, variant, or derivative thereof in combination with an agent that inhibits TGFβ. In some embodiments, the antibody blocks the interaction of SEMA4D with one or more of its receptors, e.g., Plexin-B1, Plexin-B2, and/or CD72. In some embodiments, the cancer cells express Plexin-B1, Plexin-B2, and/or CD72. In some embodiments, the stromal and/or immune cells in the tumor microenvironment express SEMA4D, Plexin-B1, Plexin-B2, and/or CD72. Anti-SEMA4D antibodies having these properties can be used in the methods provided herein. Antibodies that can be used include MAbs VX15/2503, 67, 76, 2282, VX18, and antigen-binding fragments, variants, or derivatives thereof which are fully described in US 20100285036, US 20080219971 and WO 2018/204895. Additional antibodies which can be used in the methods provided herein include the BD16 antibody described in US 20060233793 as well as antigen-binding fragments, variants, or derivatives thereof; or any of MAb 301, MAb 1893, MAb 657, MAb 1807, MAb 1656, MAb 1808, Mab 59, MAb 2191, MAb 2274, MAb 2275, MAb 2276, MAb 2277, MAb 2278, MAb 2279, MAb 2280, MAb 2281, MAb 2282, MAb 2283, MAb 2284, and MAb 2285, BD16. BB18, humanized versions thereof, as well as any fragments, variants or derivatives thereof as described in US 20080219971. In some embodiments, an anti-SEMA4D antibody for use in the methods provided herein binds human, murine, or both human and murine SEMA4D. Also useful are antibodies which bind to the same epitope as any of the aforementioned antibodies and/or antibodies which competitively inhibit binding or activity of any of the aforementioned antibodies.

In some embodiments, an anti-SEMA4D antibody or antigen-binding fragment, variant, or derivative thereof useful in the methods provided herein has an amino acid sequence that has at least about 80%, about 85%, about 88%, about 89%, about 90%, about 91%, about 92%, about 93%, about 94%, or about 95% sequence identity to the amino acid sequence for a reference anti-SEMA4D antibody molecule, for example, those described above. In a further embodiment, the binding molecule shares at least about 96%, about 97%, about 98%, about 99%, or 100% sequence identity to a reference antibody.

In some embodiments, an anti-SEMA4D antibody or antigen-binding fragment, variant, or derivative thereof useful in the methods provided herein comprises, consists essentially of, or consists of an immunoglobulin heavy chain variable domain (VH domain), where at least one of the CDRs of the VH domain has an amino acid sequence that is at least about 80%, about 85%, about 90%, about 95%, about 96%, about 97%, about 98%, about 99%, or identical to CDR1, CDR2 or CDR3 of SEQ ID NO: 9, 10, 25, 48, or 53.

In some embodiments, an anti-SEMA4D antibody or antigen-binding fragment, variant, or derivative thereof useful in the methods provided herein comprises, consists essentially of, or consists of an immunoglobulin heavy chain variable domain (VH domain), where at least one of the CDRs of the VH domain has an amino acid sequence that is at least about 80%, about 85%, about 90%, about 95%, about 96%, about 97%, about 98%, about 99%, or identical to SEQ ID NO: 6, SEQ ID NO: 7, SEQ ID NO: 8, SEQ ID NO: 26, SEQ ID NO: 27, SEQ ID NO: 28, SEQ ID NO: 54, SEQ ID NO: 55, or SEQ ID NO: 56.

In some embodiments, an anti-SEMA4D antibody or antigen-binding fragment, variant, or derivative thereof useful in the methods provided herein comprises, consists essentially of, or consists of an immunoglobulin heavy chain variable domain (VH domain), where at least one of the CDRs of the VH domain has an amino acid sequence identical, except for 1, 2, 3, 4, or 5 conservative amino acid substitutions, to SEQ ID NO: 6, SEQ ID NO: 7, SEQ ID NO: 8, SEQ ID NO: 26, SEQ ID NO: 27, SEQ ID NO: 28, SEQ ID NO: 54, SEQ ID NO: 55, or SEQ ID NO: 56.

In some embodiments, an anti-SEMA4D antibody or antigen-binding fragment, variant, or derivative thereof useful in the methods provided herein comprises, consists essentially of, or consists of a VH domain that has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 9, SEQ ID NO: 10, SEQ ID NO: 25, SEQ ID NO: 48, SEQ ID NO: 53, wherein an anti-SEMA4D antibody comprising the encoded VH domain specifically or preferentially binds to SEMA4D.

In some embodiments, an anti-SEMA4D antibody or antigen-binding fragment, variant, or derivative thereof useful in the methods provided herein comprises, consists essentially of, or consists of an immunoglobulin light chain variable domain (VL domain), where at least one of the CDRs of the VL domain has an amino acid sequence that is at least about 80%, about 85%, about 90%, about 95%, about 96%, about 97%, about 98%, about 99%, or identical to CDR1, CDR2 or CDR3 of SEQ ID NO: 17, 18, 29, or 47.

In some embodiments, an anti-SEMA4D antibody or antigen-binding fragment, variant, or derivative thereof useful in the methods provided herein comprises, consists essentially of, or consists of an immunoglobulin light chain variable domain (VL domain), where at least one of the CDRs of the VL domain has an amino acid sequence that is at least about 80%, about 85%, about 90%, about 95%, about 96%, about 97%, about 98%, about 99%, or identical to SEQ ID NO: 14, SEQ ID NO: 15, SEQ ID NO: 16, SEQ ID NO: 30, SEQ ID NO: 31, SEQ ID NO: 32, SEQ ID NO: 50, SEQ ID NO: 51, or SEQ ID NO: 52.

In some embodiments, an anti-SEMA4D antibody or antigen-binding fragment, variant, or derivative thereof useful in the methods provided herein comprises, consists essentially of, or consists of an immunoglobulin light chain variable domain (VL domain), where at least one of the CDRs of the VL domain has an amino acid sequence identical, except for 1, 2, 3, 4, or 5 conservative amino acid substitutions, to SEQ ID NO: 14, SEQ ID NO: 15, SEQ ID NO: 16, SEQ ID NO: 30, SEQ ID NO: 31, SEQ ID NO: 32, SEQ ID NO: 50, SEQ ID NO: 51, or SEQ ID NO: 52.

In a further embodiment, an anti-SEMA4D antibody or antigen-binding fragment, variant, or derivative thereof useful in the methods provided herein comprises, consists essentially of, or consists of a VL domain that has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 17, SEQ ID NO: 18, SEQ ID NO: 29, SEQ ID NO: 47, or SEQ ID NO: 49, wherein an anti-SEMA4D antibody comprising the encoded VL domain specifically or preferentially binds to SEMA4D.

Methods for measuring anti-SEMA4D binding molecule, e.g., an antibody or antigen-binding fragment, variant, or derivative thereof, binding specificity include standard competitive binding assays, assays for monitoring immunoglobulin secretion by T cells or B cells, T cell proliferation assays, apoptosis assays, ELISA assays, and the like. See, for example, such assays disclosed in WO 93/14125; Shi et al., Immunity 13: 633-642 (2000); Kumanogoh et al., J Immunol 169: 1175-1181 (2002); Watanabe et al., J Immunol 167: 4321-4328 (2001); Wang et al., Blood 97: 3498-3504 (2001); and Giraudon et al., J Immunol 172(2): 1246-1255 (2004), all of which are herein incorporated by reference.

When discussed herein whether any particular polypeptide, including the constant regions, CDRs, VH domains, or VL domains disclosed herein, is at least about 65%, about 70%, about 75%, about 80%, about 85%, about 90%, about 91%, about 92%, about 93%, about 94%, about 95%, about 96%, about 97%, about 98%, about 99%, or even about 100% identical to another polypeptide, the % identity can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman (1981) Adv Appl Math 2: 482-489, to find the best segment of homology between two sequences. When using BESTFIT or any other sequence alignment program to determine whether a particular sequence is, for example, 95% identical to a reference sequence according to the present disclosure, the parameters are set, of course, such that the percentage of identity is calculated over the full length of the reference polypeptide sequence and that gaps in homology of up to 5% of the total number of amino acids in the reference sequence are allowed.

For purposes of the present disclosure, percent sequence identity can be determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv Appl Math 2: 482-489. A variant can, for example, differ from a reference anti-SEMA4D antibody (e.g., MAb VX15/2503, 67, 76, or 2282) by as few as 1 to 15 amino acid residues, as few as 1 to 10 amino acid residues, such as 6-10, as few as 5, as few as 4, 3, 2, or even 1 amino acid residue.

The constant region of an anti-SEMA4D antibody can be mutated to alter effector function in a number of ways. For example, see U.S. Pat. No. 6,737,056 and US 20040132101, which disclose Fc mutations that optimize antibody binding to Fc receptors.

In certain anti-SEMA4D antibodies or fragments, variants or derivatives thereof useful in the methods provided herein, the Fc portion can be mutated to decrease effector function using techniques known in the art. For example, the deletion or inactivation (through point mutations or other means) of a constant region domain can reduce Fc receptor binding of the circulating modified antibody thereby increasing tumor localization. In other cases, constant region modifications consistent with the instant disclosure moderate complement binding and thus reduce the serum half-life. Yet other modifications of the constant region can be used to modify disulfide linkages or oligosaccharide moieties that allow for enhanced localization due to increased antigen specificity or antibody flexibility. The resulting physiological profile, bioavailability, and other biochemical effects of the modifications, such as tumor localization, biodistribution and serum half-life, can easily be measured and quantified using known immunological techniques without undue experimentation.

Anti-SEMA4D antibodies for use in the methods provided herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative can contain one or more non-classical amino acids.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an anti-SEMA4D polypeptide, to block SEMA4D interaction with its receptor, or to inhibit, delay, or reduce metastases in a subject, e.g., a cancer patient).

For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or improve a hybridoma's antibody production. Alternatively, non-neutral missense mutations can alter an antibody's ability to bind antigen. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein, (e.g., ability to immunospecifically bind at least one epitope of a SEMA4D polypeptide) can be determined using techniques described herein or by routinely modifying techniques known in the art.

In some embodiments, the anti-SEMA4D antibodies for use in the methods provided herein comprise at least one optimized complementarity-determining region (CDR). By “optimized CDR” is intended that the CDR has been modified and optimized to improve binding affinity and/or anti-SEMA4D activity that is imparted to an anti-SEMA4D antibody comprising the optimized CDR. “Anti-SEMA4D activity” or “SEMA4D blocking activity” can include activity which modulates one or more of the following activities associated with SEMA4D: B cell activation, aggregation and survival; CD40-induced proliferation and antibody production; antibody response to T cell dependent antigens; T cell or other immune cell proliferation; dendritic cell maturation; secretion of immune factors such as chemokines and cytokine; myeloid cell function; demyelination and axonal degeneration; apoptosis of pluripotent neural precursors and/or oligodendrocytes; induction of endothelial cell migration; inhibition of spontaneous monocyte migration; inhibition, delay, or reduction of tumor cell growth or metastasis, binding to cell surface plexin B1 or other receptor, or any other activity association with soluble SEMA4D or SEMA4D that is expressed on the surface of SEMA4D+ cells. In a particular embodiment, anti-SEMA4D activity includes the ability to inhibit, delay, or reduce tumor metastases, either in combination with inhibition, delay, or reduction of primary tumor cell growth and tumor metastases, or independently of primary tumor cell growth and tumor metastases. Anti-SEMA4D activity can also be attributed to a decrease in incidence or severity of diseases associated with SEMA4D expression, including, but not limited to, certain types of cancers including lymphomas, autoimmune diseases, inflammatory diseases including central nervous system (CNS) and peripheral nervous system (PNS) inflammatory diseases, transplant rejections, and invasive angiogenesis. Examples of optimized antibodies based on murine anti-SEMA4D MAb BD16 were described in US 20080219971, International Patent Application WO 93/14125 and Herold et al., Int Immunol 7(1): 1-8 (1995), each of which are herein incorporated by reference in their entirety. The modifications can involve replacement of amino acid residues within the CDR such that an anti-SEMA4D antibody retains specificity for the SEMA4D antigen and has improved binding affinity and/or improved anti-SEMA4D activity.

### Binding Characteristics of Anti-SEMA4D Antibodies

In some embodiments, the binding molecule is an antibody which specifically binds to SEMA4D, or an antigen-binding fragment, variant, or derivative thereof. In some embodiments, the binding molecule binds to an epitope of SEMA4D. The nucleotide and amino acid sequences for one variant of SEMA4D are set forth in SEQ ID NO: 23 and SEQ ID NO: 1, respectively. In some embodiments, the anti-SEMA4D antibody designated as VX15/2503 is provided. Antibodies that have the binding characteristics of antibody VX15/2503 are also disclosed herein. Such antibodies include antibodies that compete in competitive binding assays with VX15/2503, as well as antibodies that bind to an epitope (as defined below) capable of binding VX15/2503. In some embodiments, the anti-SEMA4D antibody designated as VX18 is provided. Antibodies that have the binding characteristics of antibody VX18 are also disclosed herein. Such antibodies include antibodies that compete in competitive binding assays with VX18, as well as antibodies that bind to an epitope (as defined below) capable of binding VX18. Methods for assessing whether antibodies have the same or similar binding characteristics include quantitative methods such as, for example, determining and comparing antibody affinity or avidity for the antigenic epitope (e.g., SEMA4D peptide). Other exemplary methods for comparing the binding characteristics of antibodies include competitive western blotting, enzyme immunoassays, ELISA, and flow cytometry. Methods for assessing and comparing antibody-antigen binding characteristics are known in the art. Variants and fragments of VX15/2503 that retain the ability to specifically bind to SEMA4D are also provided. Antibodies VX15/2503 and 67 share the same 6 CDRs and bind the same SEMA4D epitope.

As used herein, a “SEMA4D epitope” refers to the part of the SEMA4D protein to which an anti-SEMA4D antibody binds. In some embodiments, an epitope recognized by an anti-SEMA4D comprises at least 4, at least 5, at least 6, at least 7, at least 8, at least 9, at least 10, at least 15, at least 20, at least 25, or between about 15 to about 30 contiguous or non-contiguous amino acids of SEMA4D. In some embodiments, the epitope has at least 80%, 85%, 90%, 95%, or 100% identity to a target polypeptide amino acid sequence (e.g., the sequence set forth in SEQ ID NO: 42, SEQ ID NO: 44 or SEQ ID NO: 46). In some embodiments, the epitope is identical to a target polypeptide amino acid sequence (e.g., the sequence set forth in SEQ ID NO: 42, SEQ ID NO: 44, or SEQ ID NO: 46) except for 4, 3, 2, 1 or 0 amino acid substitutions. In some embodiments, the epitope is identical to a target polypeptide amino acid sequence (e.g., the sequence set forth in SEQ ID NO: 42, SEQ ID NO: 44, or SEQ ID NO: 46) except for conservative amino acid substitutions (e.g., 10, 9, 8, 7, 6, 5, 4, 3, 2, 1 or 0 conservative amino acid substitutions). In some embodiments, the epitope comprises a sequence set forth in SEQ ID NO: 42, SEQ ID NO: 44, or SEQ ID NO: 46. In some embodiments, the epitope is the sequence set forth in SEQ ID NO: 42, SEQ ID NO: 44, or SEQ ID NO: 46. In some embodiments, the epitope is a linear epitope. In some embodiments, the epitope is a conformational epitope. In some embodiments, the epitope comprises, consists essentially of, or consists of LKVPVFYALFTPQLNNV (SEQ ID NO: 42, corresponding to residues 304 through 320 of the full-length SEMA4D amino acid sequence set forth in SEQ ID NO: 1), KWTSFLKARLIASRP (SEQ ID NO: 44, corresponding to residues 270 through 284 of the full-length SEMA4D amino acid sequence set forth in SEQ ID NO: 1, wherein position 281 can be a cysteine or an alanine), or EFVFRVLIPRIARV (SEQ ID NO: 46; corresponding to residues 243 through 256 of the full-length SEMA4D amino acid sequence set forth in SEQ ID NO: 1). In some embodiments, the epitope comprises one or more of the amino acid sequences set forth in SEQ ID NO: 42, 44 and 46. In some embodiments, the epitope is a discontinuous epitope comprised in the domain spanning amino acid residues 243 to 320 of SEQ ID NO: 1.

Treatment Methods Using Therapeutic Anti-SEMA4D Antibodies in Combination with an Agent that Binds TGFβ

The use of anti-SEMA4D or anti-Plexin-B1, anti-Plexin-B2, and/or anti-CD72 binding molecules, e.g., antibodies, including antigen-binding fragments, variants, and derivatives thereof, either as single agents or in combination with at least one other immune modulating therapy, to inhibit, delay, or reduce tumor growth or metastases in a subject in need of such inhibition, delay, or reduction, e.g., a cancer patient is contemplated herein. In some embodiments, the cancer and stromal cells consisting of the tumor microenvironment express SEMA4D and/or SEMA4D receptors. In some embodiments, the receptor is Plexin-B1. In some embodiments, the receptor is Plexin-B2. In some embodiments, the receptor is CD72. Though the following discussion refers to administration of an anti-SEMA4D antibody, the methods described herein are equally applicable to the antigen-binding fragments, variants, and derivatives of these antibodies that retain the desired properties of the antibodies of the disclosure, e.g., capable of specifically binding SEMA4D, e.g., human, mouse, or human and mouse SEMA4D, having SEMA4D neutralizing activity, and/or blocking the interaction of SEMA4D with its receptors. The methods described herein are also applicable to other biologic products or small molecule drugs that retain the desired properties of the antibodies of the disclosure, e.g., capable of specifically binding SEMA4D, e.g., human, mouse, or human and mouse SEMA4D, having SEMA4D neutralizing activity, and/or blocking the interaction of SEMA4D with its receptors.

In some embodiments, anti-SEMA4D binding molecules, e.g., antibodies, including antigen-binding fragments, variants, and derivatives thereof, can be used in combination with a TGFβ inhibitor to inhibit, delay, or reduce tumor growth in a subject in need of such inhibition, delay, or reduction, e.g., a cancer patient. In some embodiments, the cancer cells express a SEMA4D receptor, such as, for example, Plexin-B1, Plexin-B2, and/or CD72. In some embodiments, the cancer cells express other receptors that can work in conjunction with a SEMA4D receptor.

In some embodiments, the treatment comprises use of anti-SEMA4D binding molecules in combination with inhibition of the TGFβ signaling pathway, and with at least one additional immune modulating therapy. In some embodiments, the immune modulating therapy can include HDAC inhibitors, cancer vaccines, immunostimulatory agents, adoptive T cell or antibody therapy, and inhibitors of immune checkpoint blockade (Lizée et al., (2013) Ann Rev Med 64: 71-90).

Inhibition of TGFβ. In some embodiments, the immune modulating therapies include those that inhibit the immunosuppressive transforming growth factor beta (TGFβ) signaling pathway. Inhibition of the TGFβ signaling pathway enhances or augments the efficacy of anti-PD1 therapies. Therefore, in some embodiments, the immune modulating therapy comprises administration of an immune checkpoint blockade therapy in addition to the inhibition of TGFβ. In some embodiments, the treatment comprises use of anti-SEMA4D binding molecules in combination with a therapeutic agent that inhibits TGFβ, alone or in combination with the inhibition of PD-1 (or PD-L1). In some embodiments, the therapeutic agent that inhibits TGFβ also inhibits PD-1 (or PD-L1). In some embodiments, the therapeutic agent is an antibody that binds TGFβ (“anti-TGFβ antibody”). In some embodiments, the anti-TGFβ antibody or antigen-binding fragment thereof is a bispecific antibody. In some embodiments, the bispecific antibody provides a combination of anti-TGFβ and an additional immune modulating therapy. Examples of antibodies that bind TGFβ are described in the following patents and publications: U.S. Pat. Nos. 5,571,714, 5,772,998, 5,783,185, 6,090,383, 6,419,928, 6,492,497, 7,151,169, 7,368,111, 7,494,651, 7,527,791, 7,723,486, 7,867,496, 7,927,593, 8,569,462, 9,145,458, 9,714,285, US 20190092846, and W02017204277. In some embodiments, the anti-TGFβ preferentially binds TGFβ1 over TGFβ2 and TGFβ3. In some embodiments, the anti-TGFβ preferentially binds TGFβ2 over TGFβ1 and TGFβ3. In some embodiments, the anti-TGFβ preferentially binds TGFβ3 over TGFβ1 and TGFβ2. In some embodiments, the therapeutic agent that inhibits PD-1 is an anti-PD-1 antibody.

Cancer Vaccines. Cancer vaccines activate the body's immune system and natural resistance to an abnormal cell, such as cancer, resulting in eradication or control of the disease. Cancer vaccines generally consist of a tumor antigen in an immunogenic formulation that activates tumor antigen-specific helper cells and/or CTLs and B cells. Vaccines can be in a variety of formulations, including, but not limited to, dendritic cells, especially autologous dendritic cells pulsed with tumor cells or tumor antigens, heterologous tumor cells transfected with an immune stimulating agent such as GM-CSF, recombinant virus, or proteins or peptides that are usually administered together with a potent immune adjuvant such as CpG.

Immunostimulatory Agents. Immunostimulatory agents act to enhance or increase the immune response to tumors, which is suppressed in many cancer patients through various mechanisms. Immune modulating therapies can target lymphocytes, macrophages, dendritic cells, natural killer cells (NK Cell), or subsets of these cells such as cytotoxic T lymphocytes (CTL) or Natural Killer T (NKT) cells. Because of interacting immune cascades, an effect on one set of immune cells will often be amplified by spreading to other cells, e.g. enhanced antigen presenting cell activity promotes response of T and B lymphocytes. Examples of immunostimulatory agents include HER2, cytokines such as G-CSF, GM-CSF and IL-2, cell membrane fractions from bacteria, glycolipids that associate with CD1d to activate Natural Killer T (NKT) cells, CpG oligonucleotides.

Macrophages and myeloid derived suppressor cells (MDSC), myelophagocytic cells of the immune system, are a fundamental part of the innate defense mechanisms, which can promote specific immunity by inducing T cell recruitment and activation. Despite this, their presence within the tumor microenvironment has been associated with enhanced tumor progression and shown to promote cancer cell growth and spread, angiogenesis and immunosuppression. Key players in the setting of their phenotype are the microenvironmental signals to which macrophages are exposed, which selectively tune their functions within a functional spectrum encompassing the M1 (tumor inhibiting macrophage) and M2 (tumor promoting macrophage) extremes. Sica et al., Seminars in Cancer Biol 18: 349-355 (2008). Increased macrophage numbers during cancer generally correlates with poor prognosis (Qualls and Murray, Curr Topics in Develop Biol 94: 309-328 (2011)). Of the multiple unique stromal cell types common to solid tumors, tumor-associated macrophages (TAMs) are significant for fostering tumor progression. Ample evidence supports a key role for MDSCs and closely related neutrophils and monocytes in immune suppression in cancer, as well as their prominent role in tumor angiogenesis, drug resistance, and promotion of tumor metastases. Gabrilovich Cancer Immunol Res. 5(1):3-8 (2017). A key role SEMA4D in modulating MDSC function and recruitment was described in Clavijo et al. Cancer Immunol Res.; 7(2):282-291 (2019). Targeting molecular pathways regulating TAM and MDSC polarization holds great promise for anticancer therapy. Ruffell et al., Trends in Immunol 33: 119-126 (2012).

Adoptive Cell Transfer. Adoptive cell transfer can employ T cell-based cytotoxic responses to attack cancer cells. Autologous T cells that have a natural or genetically engineered reactivity to a patient's cancer are generated and expanded in vitro and then transferred back into the cancer patient. One study demonstrated that adoptive transfer of in vitro expanded autologous tumor-infiltrating lymphocytes was an effective treatment for patients with metastatic melanoma. See Rosenberg et al. (2008) Nat Rev Cancer 8(4): 299-308. This can be achieved by taking T cells that are found within resected patient tumor. These T cells are referred to as tumor-infiltrating lymphocytes (TIL) and are presumed to have trafficked to the tumor because of their specificity for tumor antigens. Such T cells can be induced to multiply in vitro using high concentrations of IL-2, anti-CD3 and allo-reactive feeder cells. These T cells are then transferred back into the patient along with exogenous administration of IL-2 to further boost their anti-cancer activity. In other studies, autologous T cells have been transduced with a chimeric antigen receptor that renders them reactive to a targeted tumor antigen. See Liddy et al., Nature Med 18: 980-7, (2012); Grupp et al., New England J Med 368: 1509-18, (2013).

Other adoptive cell transfer therapies employ autologous dendritic cells exposed to natural or modified tumor antigens ex vivo that are re-infused into the patient. Provenge is such an FDA approved therapy in which autologous cells are incubated with a fusion protein of prostatic acid phosphatase and GM-CSF to treat patients with prostate tumors. GM-CSF is thought to promote the differentiation and activity of antigen presenting dendritic cells (Small et al., J Clin Oncol 18: 3894-903(2000); US 7414108)).

Immune Checkpoint Blockade Immune checkpoint blockade therapies enhance T-cell immunity by removing a negative feedback control that limits ongoing immune responses. These types of therapies target inhibitory pathways in the immune system that are crucial for modulating the duration and amplitude of physiological immune responses in peripheral tissues (anti-CTLA4) or in tumor tissue expressing PD-L1 (anti-PD1 or anti-PD-L1) in order to minimize collateral tissue damage. Tumors can evolve to exploit certain immune-checkpoint pathways as a major mechanism of immune resistance against T cells that are specific for tumor antigens. Since many immune checkpoints are initiated by ligand-receptor interactions, these checkpoints can be blocked by antibodies to either receptor or ligand or can be modulated by soluble recombinant forms of the ligands or receptors. Neutralization of immune checkpoints allows tumor-specific T cells to continue to function in the otherwise immunosuppressive tumor microenvironment. Examples of immune checkpoint blockade therapies are those which target Cytotoxic T-lymphocyte-associated antigen 4 (CTLA-4), PD-1, its ligand PD-L1, LAG3, anti-Tim3, and B7-H3.

Cyclophosphamide Cyclophosphamide, a commonly used chemotherapeutic agent, can enhance immune responses. Cyclophosphamide differentially suppresses the function of regulatory T cells (Tregs) relative to effector T cells. Tregs are important in regulating anticancer immune responses. Tumor-infiltrating Tregs have previously been associated with poor prognosis. While agents that target Tregs specifically are currently unavailable, cyclophosphamide has emerged as a clinically feasible agent that can preferentially suppress Tregs relative to other T cells and, therefore, allows more effective induction of antitumor immune responses.

Histone deacetylase (HDAC) inhibitors. Histone Deacetylases (HDACs) regulate histone acetylation and hence act as epigenetic modulating agents. HDAC inhibitors are known to affect cancer cell viability and biology and have been used in the treatment of cancer patients. The Class I HDAC inhibitor Entinostat has been shown to markedly enhance anti-tumor vaccination. WO/2018/175179 describes the use of an antibody or antigen-binding fragment thereof that specifically binds to SEMA4D, in combination with the HDACi Entinostat. In some embodiments, the treatment comprises use of anti-SEMA4D binding molecules in combination with inhibition of the TGFβ signaling pathway, and with an HDAC inhibitor. In some embodiments, the HDAC inhibitor is Entinostat.

In some embodiments, the immune modulating therapy can be administered in context with another cancer therapy, including, but not limited to, surgery or surgical procedures (e.g. splenectomy, hepatectomy, lymphadenectomy, leukophoresis, bone marrow transplantation, and the like); radiation therapy; chemotherapy, optionally in combination with autologous bone marrow transplant, or other cancer therapy; where the additional cancer therapy is administered prior to, during, or subsequent to the anti-SEMA4D binding molecule, e.g., antibody or antigen binding fragment, variant, or derivative thereof, therapy. In some embodiments, where the combined therapies comprise administration of an anti-SEMA4D binding molecule, e.g., an antibody or antigen binding fragment, variant, or derivative thereof, in combination with administration of another therapeutic agent, the methods encompass co-administration, using separate formulations or a single pharmaceutical formulation, with simultaneous or consecutive administration in either order.

In some embodiments, the disclosure is directed to the use of anti-SEMA4D binding molecules, e.g., antibodies, including antigen-binding fragments, variants, and derivatives thereof, either as single agents or in combination with at least one other immune modulating therapy, to treat cancer patients with elevated levels of either B cells, T cells or both B cells and T cells in circulation when compared to other patients with solid tumors, such as those found in the brain, ovary, breast, colon and other tissues but excluding hematological cancers. As used herein, the term “elevated” refers to cancer patients that have at least 1.5 times, e.g., about 1.5 to about 5 times, e.g., about 1.5, 2, 2.5, 3, 3.5, 4, 4.5, or 5 or more times the mean number of B cells and/or T cells in circulation than other cancer patients. For example, in a group of 34 patients with solid tumors, the mean number of B cells was 98 per microliter of blood and the mean number of T cells was 782 per microliter of blood. Accordingly, the mean number of B cells and T cells per microliter of blood observed in this subset of cancer patients with elevated B cell and T cell levels can range from about 147 to about 588 and from about 1173 to about 3910, respectively, when compared to other cancer patients.

In some embodiments, the disclosure is directed to the use of anti-SEMA4D or anti-Plexin-B1, anti-Plexin-B2, or anti-CD72 binding molecules, e.g., antibodies, including antigen-binding fragments, variants, and derivatives thereof, either as single agents or in combination with at least one other immune modulating therapy, to treat cancer patients with levels of either B cells, T cells or both B cells and T cells in circulation that fall within or above the range of normal individuals. As used herein, the term “normal” refers to the B and/or T cell levels that are found in healthy, non-cancer patients. As used herein, the term “within” refers to a ten (10) percent difference in B and/or T cell levels. For example, the range of normal levels includes a B cell count of about 250 cells per microliter or more and/or a T cell count of about 1500 cells per microliter or more. Therefore, the mean number of B cells and T cells per microliter of blood in cancer patients with elevated B cell and T cell levels can range from about 225 to about 275 or more and from about 1350 to about 1650 and more, respectively, when compared to healthy, non-cancer patients. Of course, one skilled in the art should appreciate that the levels of B and T cells can vary depending on a variety of factors, e.g., type of cancer, stage of cancer, etc., and, therefore, levels that are below the ones provided above can also constitute elevated levels for a certain type or stage of cancer.

In some embodiments, the absolute T and B cell counts are measured using a validated flow cytometric-based immunophenotypic assay (BD Mutitest 6-color TBNK Reagent), which is a six color direct immunfluorescent assay that also utilizes BD Trucount tubes and a BD FACScanto flow cytometer. This assay is used routinely to determine the percentages and absolute counts of T, B, and NK cells as well as CD4 and CD8 subpopulations of T cells in peripheral blood. Peripheral blood cells are first gated on CD45+ lymphocytes. T cells are defined as CD3+ cells within this gate and B cells are defined as CD19+ CD3− cells within this gate. Percentages are simply taken directly from the flow cytometer after the appropriate gate is set, and the absolute counts are calculated using the following formula (taken directly from the BD procedure manual): [(#events in cell population/#events in absolute count bead region)]* [(#beads/testa)/test volume]=cell population absolute count, where “a” is the value found on the BD Trucount tube foil pouch label.

It should also be appreciated that the methods described herein are also applicable to the substitution of anti-Plexin-B1, anti-Plexin-B2, and/or anti-CD72 binding molecules for anti-SEMA4D binding molecules. In some embodiments, for example, an anti-Plexin-B1, anti-Plexin-B2, and/or anti-CD72binding molecule can be used to inhibit the interaction of SEMA4D with Plexin-B1, Plexin-B2, or CD72 by blocking binding of SEMA4D to Plexin-B1, Plexin-B2, CD72, and/or by preventing activation of Plexin-B1, Plexin-B2, or CD72 by SEMA4D. It should also be appreciated that the methods described herein are also applicable to the use of small molecule drugs or other biologic products to inhibit the activity of SEMA4D or Plexin-B1, Plexin-B2, and/or CD72. In some embodiments, a small molecule drug or a biologic product other than an anti-SEMA4D binding molecule can be used to inhibit the interaction of SEMA4D with Plexin-B1, Plexin-B2, or CD72 by blocking binding of SEMA4D to Plexin-B1, Plexin-B2, or CD72 and/or by preventing activation of Plexin-B1, Plexin-B2, or CD72 by SEMA4D.

In some embodiments, treatment includes the application or administration of an anti-SEMA4D binding molecule, e.g., an antibody or antigen binding fragment thereof as described herein in combination with an agent that inhibits TGFβ, e.g., an anti-TGFβ antibody or antigen-binding fragment thereof to a patient, or application or administration of the anti-SEMA4D binding molecule in combination with an agent that inhibits TGFβ, e.g., an anti-TGFβ antibody or antigen-binding fragment thereof to an isolated tissue or cell line from a patient, where the patient has, or has the risk of developing metastases of cancer cells. In some embodiments, treatment is also intended to include the application or administration of a pharmaceutical composition comprising the anti-SEMA4D binding molecules, e.g., an antibody or antigen binding fragment thereof to a patient, in combination with an agent that inhibits TGFβ, e.g., an anti-TGFβ antibody or antigen-binding fragment thereof or application or administration of a pharmaceutical composition comprising the anti-SEMA4D binding molecule and an agent that inhibits TGFβ, e.g., an anti-TGFβ antibody or antigen-binding fragment thereof to an isolated tissue or cell line from a patient, where the patient has, or has the risk of developing metastases of cancer cells. Administration of the combination of anti-SEMA4D and anti-TGF agents results in an enhanced therapeutic efficacy relative to treatment with either agent alone.

The anti-SEMA4D binding molecules, e.g., antibodies or binding fragments thereof as described herein, in combination with an agent that inhibits TGFβ, e.g., an anti-TGFβ antibody or antigen-binding fragment thereof are useful for the treatment of various malignant and non-malignant tumors. By “anti-tumor activity” is intended a reduction in the rate of SEMA4D production or accumulation associated directly with the tumor or indirectly with stromal cells of the tumor environment, and hence a decline in growth rate of an existing tumor or of a tumor that arises during therapy, and/or destruction of existing neoplastic (tumor) cells or newly formed neoplastic cells, and hence a decrease in the overall size of a tumor and/or the number of metastatic sites during therapy. For example, therapy with at least one anti-SEMA4D antibody in combination with an agent that inhibits TGFβ, e.g., an anti-TGFβ antibody or antigen-binding fragment thereof causes a physiological response, for example, a reduction in metastases, that is beneficial with respect to treatment of disease states associated with SEMA4D -expressing cells in a human.

In some embodiments, the disclosure relates to the use of anti-SEMA4D binding molecules, e.g., antibodies or antigen-binding fragments, variants, or derivatives thereof, in combination with an agent that inhibits TGFβ, e.g., an anti-TGFβ antibody or antigen-binding fragment thereof as a medicament, in the treatment or prophylaxis of cancer or for use in a precancerous condition or lesion to inhibit, reduce, prevent, delay, or minimalize the growth or metastases of tumor cells.

In accordance with the methods of the present disclosure, at least one anti-SEMA4D binding molecule, e.g., an antibody or antigen binding fragment, variant, or derivative thereof, in combination with an agent that inhibits TGFβ, e.g., an anti-TGFβ antibody or antigen-binding fragment thereof can be used to promote a positive therapeutic response with respect to a malignant human cell. By “positive therapeutic response” with respect to cancer treatment is intended an improvement in the disease in association with the anti-tumor activity of these binding molecules, e.g., antibodies or fragments thereof, and/or an improvement in the symptoms associated with the disease. In particular, the methods provided herein are directed to inhibiting, preventing, reducing, alleviating, delaying, or lessening growth of a tumor and/or the development of metastases of primary tumors in a patient. That is the prevention of distal tumor outgrowths, can be observed. Thus, for example, an improvement in the disease can be characterized as a complete response. By “complete response” is intended an absence of clinically detectable metastases with normalization of any previously abnormal radiographic studies, e.g. at the site of the primary tumor or the presence of tumor metastases in bone marrow. Alternatively, an improvement in the disease can be categorized as being a partial response. By “partial response” is intended at least about a 50% decrease in all measurable metastases (i.e., the number of tumor cells present in the subject at a remote site from the primary tumor). Alternatively, an improvement in the disease can be categorized as being relapse free survival or “progression free survival”. By “relapse free survival” is intended the time to recurrence of a tumor at any site. “Progression free survival” is the time before further growth of tumor at a site being monitored can be detected.

Inhibition, delay, or reduction of metastases can be assessed using screening techniques such as imaging, for example, fluorescent antibody imaging, bone scan imaging, and tumor biopsy sampling including bone marrow aspiration (BMA), or immunohistochemistry. In addition to these positive therapeutic responses, the subject undergoing therapy with the anti-SEMA4D binding molecule, e.g., an antibody or antigen-binding fragment, variant, or derivative thereof, can experience the beneficial effect of an improvement in the symptoms associated with the disease. The beneficial effect of anti-SEMA4D therapy when combined with anti-TGFβ therapy is greater than the benefit of either therapy alone.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like.

### Methods of Diagnosis and Treatment

In some embodiments, this disclosure provides methods of treating a subject, e.g., a cancer patient, where the subject has elevated levels of either B cells, T cells or both B cells and T cells, comprising administering a combination of an effective amount of an isolated binding molecule that specifically binds to semaphorin-4D (SEMA4D) and an effective amount of an agent that inhibits TGFβ, e.g., an anti-TGFβ antibody or antigen-binding fragment thereof if the subject's B cell, T cell or both B cell and T cell levels are above a predetermined threshold level of B cells, T cells or both B cells and T cells, or are elevated relative to the level of B cells, T cells or both B cells and T cells, in one or more control samples that can include samples from other cancer patients or from healthy, non-cancer patients. B cell, T cell, or B cell and T cell levels can be measured by a healthcare provider or by a clinical laboratory, where a sample, e.g., a blood sample, is obtained from the patient either by the healthcare provider or by the clinical laboratory. In one aspect, the patient's level of B cells, T cells or both B cells and T cells, can be measured in a cytometric-based immunophenotypic assay.

In some embodiments, this disclosure also provides a method of treating a subject, e.g., a cancer patient, comprising administering to the subject an effective amount of an isolated binding molecule that specifically binds to semaphorin-4D (SEMA4D) and an effective amount of an agent that inhibits TGFβ, e.g., an anti-TGFβ antibody or antigen-binding fragment thereof if Her2 and either Plexin B1 or Plexin B2 expression in a sample taken from the subject's tumor cells is above predetermined threshold levels, or is elevated relative to the Her2 and either Plexin B1 or Plexin B2 expression in one or more control samples. Her2, Plexin B1, and/or Plexin B2 expression in the subject's tumor or immune cells can be measured by a healthcare provider or by a clinical laboratory at the protein level and/or at the mRNA level. In some embodiments, Her2, Plexin B1, and/or Plexin B2 expression can be measured in situ, e.g., via imaging techniques. In some embodiments, Her2, Plexin B1, and/or Plexin B2 expression can be measured in a tumor cell sample obtained from the subject via a biopsy. In one aspect, Her2, Plexin B1, and/or Plexin B2 expression in tumor cells can be measured in an immunoassay employing antibodies or antigen binding fragments thereof which recognize Her2, Plexin B1, and/or Plexin B2 proteins, or antigen-binding fragments, variants or derivatives thereof. In another aspect Her2, Plexin B1, and/or Plexin B2 expression can be measured via a quantitative gene expression assay, e.g., an RT-PCR assay.

This disclosure also provides methods, assays, and kits to facilitate a determination by a healthcare provider, a healthcare benefits provider, or a clinical laboratory to as to whether a subject, e.g., a cancer patient, will benefit from treatment with an effective amount of an isolated binding molecule that specifically binds to semaphorin-4D (SEMA4D) and an effective amount of an agent that inhibits TGFβ, e.g., an anti-TGFβ antibody or antigen-binding fragment thereof, where the subject has, or is suspected to have, tumor cells that are Her2+and either Plexin B1+or Plexin B2+. The methods, assays, and kits provided herein will also facilitate a determination by a healthcare provider, a healthcare benefits provider, or a clinical laboratory to as to whether a subject, e.g., a cancer patient, will benefit from treatment with an effective amount of an isolated binding molecule that specifically binds to semaphorin-4D (SEMA4D) and an effective amount of an agent that inhibits TGFβ, e.g., an anti-TGFβ antibody or antigen-binding fragment thereof (e.g., where the subject's tumor cells express, or can be determined to express, Her2 and either Plexin B1 or Plexin B2),.

The present disclosure provides a method of treating a subject, e.g., a cancer patient, comprising administering an effective amount of an isolated binding molecule that specifically binds to semaphorin-4D (SEMA4D) and an effective amount of an agent that inhibits TGFβ, e.g., an anti-TGFβ antibody or antigen-binding fragment thereof if the level of B-cells, T-cells, or T-cells and B-cells in a sample taken from the patient is above a predetermined threshold level, or is above the level of B-cells, T-cells, or T-cells and B-cells in one or more control samples. In some aspects, the sample is obtained from the patient and is submitted for measurement of the level of B-cells, T-cells, or T-cells and B-cells in the sample, for example, to a clinical laboratory.

Also provided is a method of treating a subject, e.g., a cancer patient, s comprising (a) submitting a sample taken from the subject for measurement of the level of B-cells, T-cells, or T-cells and B-cells in the sample; and, (b) administering an effective amount of an isolated binding molecule that specifically binds to semaphorin-4D (SEMA4D) and an effective amount of an agent that inhibits TGFβ, e.g., an anti-TGFβ antibody or antigen-binding fragment thereof to the subject if the subject's level of B-cells, T-cells, or T-cells and B-cells is above a predetermined threshold level, or is above the level of B-cells, T-cells, or T-cells and B-cells in one or more control samples.

The disclosure also provides a method of treating a subject, e.g., a cancer patient, comprising (a) measuring the level of B-cells, T-cells, or T-cells and B-cells in a sample obtained from a subject, e.g., a cancer patient, wherein the subject's level of B-cells, T-cells, or T-cells and B-cells in the sample is measured, e.g., in a cytometric-based immunophenotypic assay; (b) determining whether the level of B-cells, T-cells, or T-cells and B-cells in the sample is above a predetermined threshold level, or is above the level of B-cells, T-cells, or T-cells and B-cells in one or more control samples; and, (c) advising, instructing, or authorizing a healthcare provider to administer an effective amount of an isolated binding molecule that specifically binds to semaphorin-4D (SEMA4D) and an effective amount of an agent that inhibits TGFβ, e.g., an anti-TGFβ antibody or antigen-binding fragment thereof to the subject if the subject's level of B-cells, T-cells, or T-cells and B-cells is above a predetermined threshold level, or is above the level of B-cells, T-cells, or T-cells and B-cells in one or more control samples.

In some aspects, the subject's level of B-cells, T-cells, or T-cells and B-cells can be measured in a cytometric-based immunophenotypic assay. In some embodiments, the assay can be performed on a sample obtained from the subject, by the healthcare professional treating the patient, e.g., using an assay as described herein, formulated as a “point of care” diagnostic kit. In some aspects, a sample can be obtained from the subject and can be submitted, e.g., to a clinical laboratory, for measurement of the level of B-cells, T-cells, or T-cells and B-cells in the sample according to the healthcare professional's instructions, including but not limited to, using a cytometric-based immunophenotypic assay as described herein. In some embodiments, the clinical laboratory performing the assay can advise the healthcare provider or a healthcare benefits provider as to whether the subject can benefit from treatment with an effective amount of an isolated binding molecule that specifically binds to semaphorin-4D (SEMA4D) and an effective amount of an agent that inhibits TGFβ, e.g., an anti-TGFβ antibody or antigen-binding fragment thereof, if the subject's level of B-cells, T-cells, or T-cells and B-cells is above a predetermined threshold level, or is above the level of B-cells, T-cells, or T-cells and B-cells in one or more control samples.

In some embodiments, results of an immunoassay as provided herein can be submitted to a healthcare benefits provider for determination of whether the patient's insurance will cover treatment with an isolated binding molecule which specifically binds to semaphorin-4D (SEMA4D) and an agent that inhibits TGFβ, e.g., an anti-TGFβ antibody or antigen-binding fragment thereof. In some embodiments, the anti-TGFβ antibody provides a combination of anti-TGFβ and an additional immune modulating therapy.

### Pharmaceutical Compositions and Administration Methods

Methods of preparing and administering anti-SEMA4D binding molecules, e.g., antibodies, or antigen-binding fragments, variants, or derivatives thereof as a single agent or in combination with an agent that inhibits TGFβ, e.g., an anti-TGFβ antibody or antigen-binding fragment thereof to a subject in need thereof are known to or are readily determined by those skilled in the art. The route of administration of the anti-SEMA4D binding molecule, e.g, antibody, or antigen-binding fragment, variant, or derivative thereof as a single agent or in combination with an agent that inhibits TGFβ, e.g., an anti-TGFβ antibody or antigen-binding fragment thereof, can be, for example, oral, parenteral, by inhalation or topical at the same or different times for each therapeutic agent. The term parenteral as used herein includes, e.g., intravenous, intraarterial, intraperitoneal, intramuscular, subcutaneous, rectal, or vaginal administration. While all these forms of administration are clearly contemplated as being within the scope of the disclosure, an example of a form for administration would be a solution for injection, in particular for intravenous or intraarterial injection or drip. A suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, anti-SEMA4D binding molecules, e.g., antibodies, or antigen-binding fragments, variants, or derivatives thereof as a single agent or in combination with at least one other immune modulating therapy can be delivered directly to the site of the adverse cellular population thereby increasing the exposure of the diseased tissue to the therapeutic agent.

As discussed herein, anti-SEMA4D binding molecules, e.g., antibodies, or antigen-binding fragments, variants, or derivatives thereof as a single agent or in combination with an agent that inhibits TGFβ, e.g., an anti-TGFβ antibody or antigen-binding fragment thereof can be administered in a pharmaceutically effective amount for the in vivo treatment of diseases such as neoplastic disorders, including solid tumors. In this regard, it will be appreciated that the disclosed binding molecules can be formulated so as to facilitate administration and promote stability of the active agent. In some embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of an anti-SEMA4D binding molecules, e.g., an antibody, or antigen-binding fragment, variant, or derivative thereof, as a single agent or in combination with an agent that inhibits TGFβ, e.g., an anti-TGFβ antibody or antigen-binding fragment thereof shall be held to mean an amount sufficient to achieve effective binding to a target and to achieve a benefit, i.e., to inhibit, delay, or reduce metastases in a cancer patient.

The pharmaceutical compositions used in this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. Pharmaceutically acceptable carriers can include 0.01-0.1 M, or 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as, for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition can be sterile and should be fluid to the extent that easy syringability exists. It should be stable under the conditions of manufacture and storage and can be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of a certain particle size in the case of dispersion and by the use of surfactants. Suitable formulations for use in the therapeutic methods disclosed herein are described in Remington's Pharmaceutical Sciences (Mack Publishing Co.) 16th ed. (1980).

Prevention of the action of microorganisms can be achieved by various antibacterial and antifungal agents, for example, parabens, chlorobutanol, phenol, ascorbic acid, thimerosal and the like. In some embodiments, isotonic agents, for example, sugars, polyalcohols, such as mannitol, sorbitol, or sodium chloride can be included in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an anti-SEMA4D antibody, or antigen-binding fragment, variant, or derivative thereof, by itself or in combination with an agent that inhibits TGFβ, e.g., an anti-TGFβ antibody or antigen-binding fragment thereof) in a certain amount in an appropriate solvent with one or a combination of ingredients enumerated herein, followed by filtered sterilization. Generally, dispersions are prepared by incorporating the active compound into a sterile vehicle, which contains a basic dispersion medium and the other ingredients from those enumerated above. In the case of sterile powders for the preparation of sterile injectable solutions, methods of preparation can include vacuum drying or freeze-drying, which can yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture can have labels or package inserts indicating that the associated compositions are useful for treating a subject suffering from or predisposed to a disease or disorder.

Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of an anti-SEMA4D binding molecule, e.g., antibody, or fragment, variant, or derivative thereof in combination with an agent that inhibits TGFβ, e.g., an anti-TGFβ antibody or antigen-binding fragment thereof to be combined with the carrier materials to produce a single dosage form will vary depending upon the host treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, anti-SEMA4D antibodies, or antigen-binding fragments, variants, or derivatives thereof in combination with an agent that inhibits TGFβ, e.g., an anti-TGFβ antibody or antigen-binding fragment thereof can be administered to a human or other animal in accordance with the aforementioned methods of treatment in an amount sufficient to produce a therapeutic effect. The anti-SEMA4D antibodies, or antigen-binding fragments, variants or derivatives thereof in combination with an agent that inhibits TGFβ, e.g., an anti-TGFβ antibody or antigen-binding fragment thereof can be administered to such human or other animal in a conventional dosage form prepared by combining the antibody provided herein with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of anti-SEMA4D binding molecules, e.g., antibodies, or antigen-binding fragments, variants, or derivatives thereof as provided herein can be used.

By “therapeutically effective dose or amount” or “effective amount” is intended an amount of anti-SEMA4D binding molecule, e.g., antibody or antigen binding fragment, variant, or derivative thereof in combination with an agent that inhibits TGFβ, e.g., an anti-TGFβ antibody or antigen-binding fragment thereof that when administered brings about a positive therapeutic response with respect to treatment of a patient with a disease to be treated, e.g., an inhibition, delay, or reduction of metastases in the patient.

Therapeutically effective doses of the compositions of the present disclosure, for the inhibition, delay, or reduction of metastases, vary depending upon many different factors, including means of administration, target site, physiological state of the patient, whether the patient is human or an animal, other medications administered, and whether treatment is prophylactic or therapeutic. In some embodiments, the patient is a human, but non-human mammals including transgenic mammals can also be treated. Treatment dosages can be titrated using routine methods known to those of skill in the art to optimize safety and efficacy.

The amount of anti-SEMA4D binding molecule, e.g., antibody or binding fragment, variant, or derivative thereof, administered a in combination with an agent that inhibits TGFβ, e.g., an anti-TGFβ antibody or antigen-binding fragment thereof is readily determined by one of ordinary skill in the art without undue experimentation given the disclosure of the present disclosure. Factors influencing the mode of administration and the respective amount of anti-SEMA4D binding molecule, e.g., antibody, antigen-binding fragment, variant or derivative thereof to be administered in combination with a an agent that inhibits TGFβ, e.g., an anti-TGFβ antibody or antigen-binding fragment thereof include the severity of the disease, the history of the disease, the potential for metastases, and the age, height, weight, health, and physical condition of the individual undergoing therapy. Similarly, the amount of anti-SEMA4D binding molecule, e.g., antibody, or fragment, variant, or derivative thereof in combination with an agent that inhibits TGFβ, e.g., an anti-TGFβ antibody or antigen-binding fragment thereof to be administered will be dependent upon the mode of administration and whether the subject will undergo a single dose or multiple doses of this agent.

The disclosure also provides for the use of an anti-SEMA4D binding molecule, e.g., antibody, or antigen-binding fragment, variant, or derivative thereof in combination with an agent that inhibits TGFβ, e.g., an anti-TGFβ antibody or antigen-binding fragment thereof in the manufacture of a medicament for treating a subject with a cancer, wherein the medicament is used in a subject that has been pretreated with at least one other therapy. By “pretreated” or “pretreatment” is intended the subject has received one or more other therapies (e.g., been treated with at least one other cancer therapy) prior to receiving the medicament comprising the anti-SEMA4D binding molecule, e.g., antibody or antigen-binding fragment, variant, or derivative thereof in combination with an agent that inhibits TGFβ, e.g., an anti-TGFβ antibody or antigen-binding fragment thereof “Pretreated” or “pretreatment” includes subjects that have been treated with at least one other therapy within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or even within 1 day prior to initiation of treatment with the medicament comprising the anti-SEMA4D binding molecule, for example, the monoclonal antibody VX15/2503 disclosed herein, or antigen-binding fragment, variant, or derivative thereof as a single agent or in combination with an agent that inhibits TGFβ, e.g., an anti-TGFβ antibody or antigen-binding fragment thereof. It is not necessary that the subject was a responder to pretreatment with the prior therapy or therapies. Thus, the subject that receives the medicament comprising the anti-SEMA4D binding molecule, e.g., an antibody or antigen-binding fragment, variant, or derivative thereof in combination with an agent that inhibits TGFβ, e.g., an anti-TGFβ antibody or antigen-binding fragment thereof could have responded, or could have failed to respond (e.g., the cancer was refractory), to pretreatment with the prior therapy, or to one or more of the prior therapies where pretreatment comprised multiple therapies. Examples of other cancer therapies for which a subject can have received pretreatment prior to receiving the medicament comprising the anti-SEMA4D binding molecule, e.g., antibody or antigen-binding fragment, variant, or derivative thereof in combination with an agent that inhibits TGFβ, e.g., an anti-TGFβ antibody or antigen-binding fragment thereof include surgery; radiation therapy; chemotherapy, optionally in combination with autologous bone marrow transplant, where suitable chemotherapeutic agents include those listed herein above; other anti-cancer monoclonal antibody therapy; small molecule-based cancer therapy, including, but not limited to, the small molecules listed herein above;

vaccine/immunotherapy-based cancer therapies; steroid therapy; other cancer therapy; or any combination thereof.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B.D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S.C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W.R., and L.M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevere, Amsterdam); Goldsby et al., eds. (2000) Kuby Immunnology (4th ed.; H. Freemand & Co.); Roitt et al., (2001) Immunology (6th ed.; London: Mosby); Abbas et al., (2005) Cellular and Molecular Immunology (5th ed.; Elsevier Health Sciences Division); Kontermann and Dubel (2001) Antibody Engineering (Springer Verlan); Sambrook and Russell (2001) Molecular Cloning: A Laboratory Manual (Cold Spring Harbor Press); Lewin (2003) Genes VIII (Prentice Hal12003); Harlow and Lane (1988) Antibodies: A Laboratory Manual (Cold Spring Harbor Press); Dieffenbach and Dveksler (2003) PCR Primer (Cold Spring Harbor Press).

All of the references cited above, as well as all references cited herein, are incorporated herein by reference in their entireties.

The following examples are offered by way of illustration and not by way of limitation.

### EXAMPLES

### Example 1: Testing the Ability of an Anti-SEMA4D Antibody to Delay Tumor Growth in Immune Competent Mice

Experimental Design. The basic experimental design is as follows. Colon26 tumor cells were implanted subcutaneously into the flank of syngeneic immunocompetent Balb/c mice (5×10⁵ cells) or immune deficient SCID mice (1×10⁵ cells) in 0.2 ml saline. Treatment with Control Ig 2B8 or anti-SEMA4D Ab 67 was initiated on day 2 post tumor implant. Mice (n=20) were treated twice weekly with 1.0 mg (approximately 50 mg/kg) each of monoclonal antibody via intraperitoneal (IP) injection. Tumors were measured with calipers 3×/week starting 3 days post implant. Mice were weighed 2×/wk starting on day 3. Animals were sacrificed when tumor volume reached 1000 mm³.

Anti-SEMA4D treatment delayed tumor growth in mice with competent immune system. Tumor growth was measured by calipers and measurements were used to calculate tumor volume using the formula (w²×l)/2, where w=width, smaller measurement and l=length, in mm, of the tumor. Twenty-nine percent (29%) tumor growth delay was achieved in Balb/c mice, however, no treatment related tumor growth delay was observed in SCID mice. Tumor growth delay (TGD), is defined as the increase in the median time-to-endpoint (TTE) in a treatment group compared to the control group: % TGD—[(T−C)/C]×100, where T=median TTE for a treatment group, C=median TTE for the control group. The Balb/c animals treated with anti-SEMA4D antibody 67 showed a statistically significant reduction in primary tumor volume at the time of sacrifice over the control animals (P<0.0001). This finding shows that the anti-SEMA4D antibody was effective at delaying tumor growth in mice with a competent immune system, but not in immune deficient mice. Details of this Example can be found in Evans EE, et al., Antibody Blockade of Semaphorin 4D Promotes Immune Infiltration into Tumor and Enhances Response to Other Immunomodulatory Therapies. Cancer Immunol Res. 2015 Jun; 3(6):689-701, and also in, for example, U.S. Pat. Nos. 9,243,068 and 9,828,435.

### Example 2: Testing the Ability of an Anti-SEMA4D Antibody to Delay Tumor Growth in Presence of CD8+ Effector T Cells

Experimental Design. Colon26 tumor cells were implanted subcutaneously into the flank of Balb/c mice (5×10⁵ cells in 0.2 ml saline). Anti-CD8 depleting antibody (Clone 2.43, BioXCell) or control Rat Ig (Clone LTF-2, BioXCell) (150 mg/kg) were administered via intraperitoneal (IP) injection on days −1, 0, 1, 11 and weekly thereafter. Treatment with Control Ig 2B8 or anti-SEMA4D Ab 67 was initiated on day 2. Mice (n=20) were treated twice weekly with 1.0 mg (approximately 50 mg/kg) of monoclonal antibody via intraperitoneal injection. Tumors were measured with calipers 3×/week starting 3 days post implant. Animals were sacrificed when the mean tumor volume of the control group reached 1000 mm³, day 30 for Rat Ig treated groups, and day 26 for anti-CD8 treated groups.

Anti-SEMA4D treatment delayed tumor growth in presence of CD8+ T lymphocytes. Tumor volume was measured by calipers using the formula (w²×l)/2, where w=width, smaller measurement and l=length, in mm, of the tumor. Statistical differences in tumor volume were determined using a two-tailed One-Way Analysis of Variance (ANOVA) comparing antibody treated groups with the Control Ig 2B8 group.

Inhibition of tumor growth was also determined. Tumor growth inhibition (TGI) was measured using the following formula: % TGI=l−[(Tf−Ti)/mean(Cf−Ci)]; % TGI reported is the mean of % TGI for each treated tumor. Statistical differences in tumor volume were determined using a two-tailed One-Way Analysis of Variance (ANOVA) followed by the Dunnett's multiple comparisons test comparing treated groups with control 2B8 group. Thirty percent (30%) tumor growth inhibition was achieved following treatment with anti-SEMA4D antibody, however no treatment related effect was observed when CD8+ T cells were depleted. These results show that tumor growth inhibition with anti-SEMA4D was dependent on the presence of CD8+ effector T cells. Details of this Example can be found in Evans EE, et al., Antibody Blockade of Semaphorin 4D Promotes Immune Infiltration into Tumor and Enhances Response to Other Immunomodulatory Therapies. Cancer Immunol Res. 2015 Jun; 3(6):689-701, and also in, for example, U.S. Pat. Nos. 9,243,068 and 9,828,435.

### Example 3: Testing the Ability of an Anti-SEMA4D Antibody to Increase Density of Tumor Infiltrating Lymphocytes (TIL)

Experimental Design. Colon26 tumor cells were implanted subcutaneously into a flank of Balb/c mice (5×10⁵ cells in 0.2 ml saline). Treatment with Control Ig 2B8 or anti-SEMA4D Ab 67 was initiated on day 2 (50 mg/kg IP, twice weekly, n=10). Tumors were measured with calipers 3×/week starting 3 days post implant. Animals were sacrificed on day 27, when the mean tumor volume of the control group reached 1000 mm³. Tumors, including surrounding stroma and skin, were collected and fixed in formalin for 24 hours, then transferred to 70% ethanol. Samples were then processed for paraffin embedding, and 5 micron sections were cut from the resulting blocks.

Adjacent sections were stained for Sema4D, CD8, and CD20 using the following methods:
a. For Sema4D detection, slides were baked at 60 ° C. for 1 hour, then deparaffinized and rehydrated through xylene and graded ethanol baths. Epitope retrieval was carried out by boiling 20-minute with Target Retrieval Solution (Dako, Carpinteria, Calif.) followed by 30-minute cooling. Slides were washed twice with PBS containing 0.05% Tween-20 (TPBS), then endogenous peroxidases were inactivated with a 10-minute block with Dual Enzyme Block (Dako, Carpinteria, Calif.). Slides were washed with TPBS twice, then nonspecific binding was blocked by a 20-minute incubation with 2.5% normal goat serum in TPBS. Following a single TPBS wash, slides were incubated for 60 minute with rabbit anti-Sema4D at 2 μg/ml in TPBS, followed by 2 TPBS washes. Slides were then incubated for 20 minute with Envision HRP labeled goat anti-rabbit polymer (Dako, Carpinteria, Calif.) followed by 2 washes with TPBS and a 5-minute DAB+incubation (Dako, Carpinteria, Calif.). Sections were counterstained with Harris hematoxylin, destained, blued with tap water, dehydrated, and non-aqueous mounted with Permount.
b. CD8 was detected using the method above, but using a commercial rabbit polyclonal antibody (Abbiotec) at 2 μg/ml.
c. CD20 was detected using the method above, but using normal donkey serum for blocking, and using a goat anti-CD20 primary antibody (Santa Cruz) at 1 μg/ml followed by a 20-minute incubation with HRP-labeled anti-goat antibody (Golden Bridge).
d. Slides were imaged at 20×magnification using a Retiga QICAM-12 bit camera coupled to an Olympus I×50 microscope.

Anti-SEMA4D treatment increased frequency of tumor infiltrating immune cells (TIL). Immune cell density was measured by scanning sections of the entire tumor, quantifying areas of CD8+ or CD20+ Tumor Infiltrating Lymphocytes (TIL), and then normalizing to total tumor area. Sections from 9 (Control Ig) or 10 (anti-SEMA4D Ab 67) mice per group were used for analysis. Statistical significance was calculated for CD8 and CD20 using two tailed unpaired T test to 95% CI.

Treatment of Colon26 tumors with anti-SEMA4D antibody 67 resulted in an increase in both CD8+ T cell density and CD20+ T cell density, as compared to the control group. The increase in density of the CD20+ T cells was statistically significant to 95% with a P value of 0.0388. The increase in density of the CD8+ T cells showed a trend but was not statistically significant. These findings show that anti-SEMA4D treatment of Colon26 tumors resulted in increased frequency of tumor infiltrating immune cells. Details of this Example can be found in Evans EE, et al., Antibody Blockade of Semaphorin 4D Promotes Immune Infiltration into Tumor and Enhances Response to Other Immunomodulatory Therapies. Cancer Immunol Res. 2015 Jun; 3(6):689-701, and also in, for example, U.S. Pat. Nos. 9,243,068 and 9,828,435.

### Example 4: Testing the Ability of an Anti-SEMA4D Antibody to Affect Migration and Distribution of M1 and M2 Macrophage Subsets and CD8+ T Cells at Leading Edge of Tumor

Anti-SEMA4D treatment altered macrophage and CD8+ T cell distribution at leading edge of tumor. Macrophage distribution was measured by scanning sections of the entire tumor, quantitating the area of M1 (staining with Alexa647 conjugated rat anti-F4/80 (Biolegend, clone BM8) at 2 μg/m1) and M2 (staining with biotin conjugated rat anti-CD206 (Biolegend, clone C068C2) at 2 μg/m1), and then normalizing to total tumor area to determine M1 and M2 density within the tumor. Sections from 9 (Control Ig) or 10 (anti-SEMA4D Ab 67) treated mice per group were used for analysis. For determining cell density in the tumor growing front, a 300 pixel width region (250 micron) was defined from the edge of the tumor. Statistical significance for M1 and M2 were calculated using one way ANOVA with Kruskal-Wallace and Dunn's post-hoc test to 95% CI. Change in density of M1 macrophage normalized to leading edge of tumor was significant.

CD8+ T cell numbers were measured in whole tumor sections stained with anti CD8 antibody (Abbiotec Cat#250596 at 1:250) and DAB detection system. The number of CD8+ events in entire tumor sections were enumerated after thresholding for positive signal using Imagepro Software. CD8+ density for each animal was calculated by dividing the number of CD8+ events by the whole tumor pixel area. Individual CD8 densities were averaged to arrive at CD8+ T cell distribution in 2B8 and mAb67 treated animals (n=10). Statistical significance was calculated using one way ANOVA with Kruskal-Wallace and Dunn's post hoc test to 95% CI.

SEMA4D distribution was measured by scanning sections of the entire tumor stained for SEMA4D with an antibody to an epitope distinct from that recognized by Ab 67 and analyzing for Sema4D distribution. Sections from 9 (Control Ig) or 10 (anti-SEMA4D Ab 67) treated mice per group were used for analysis.

Colon26 tumor cells expressed low levels of SEMA4D when cultured in vitro, but upregulated SEMA4D in vivo at the leading edge of the tumor. This led to establishment of a gradient of SEMA4D expression with high concentration at the periphery of the tumor. Treatment with anti-SEMA4D antibody neutralized SEMA4D and disrupted the gradient of expression. This resulted in a striking change in the migration and distribution of macrophage. In particular, tumors treated with anti-SEMA4D Ab 67 had higher levels of M1+ pro-inflammatory macrophages at the leading edge to the tumor. The increase in M1+ macrophage was statistically significant. Tumors treated with anti-SEMA4D Ab 67 also showed a decrease in the frequency of pro-tumor M2 macrophage at the leading edge of the tumor. These findings showed that treatment with anti-SEMA4D Ab 67 altered macrophage distribution in a way that increased the density of tumor inhibitory macrophage, i.e., Ml, at the leading edge of the tumor while decreasing the presence of tumor promoting macrophage, i.e., M2, in that same region. Furthermore, these findings showed an overall increase in the CD8+ T cell density within tumors isolated from MAb 67-treated mice. These findings suggest that neutralization of SEMA4D with MAb 67-2 facilitates entry of anti-tumor M1 macrophage into the zone of highly proliferating tumor cells and CD8+ T cells throughout the zone and extending into the leading edge. Further details of this Example can be found in Evans EE, et al., Antibody Blockade of Semaphorin 4D Promotes Immune Infiltration into Tumor and Enhances Response to Other Immunomodulatory Therapies. Cancer Immunol Res. 2015 Jun; 3(6):689-701. Additional data showing T cell infiltration into a 3rd mouse tumor model (MOC1 head and neck tumor) treated with anti-SEMA4D can be found in Clavijo PE, et al., Semaphorin4D inhibition improves response to immune checkpoint blockade via attenuation of MDSC recruitment and function. Cancer Immunol Res. 2019 Feb; 7(2):282-291 (see FIG. 6A-B therein).

### Example 5: Testing the Ability of an Anti-SEMA4D Antibody to Affect Tumor Infiltration of Tumor-Specific Cytotoxic CD8+ T Cells and Secretion of Cytokines and Chemokines.

MAb 67-2 treatment increases the frequency of tumor-specific TIL. Following four weeks of in-vivo anti-SEMA4D treatment, tumors were dissociated and enriched for CD45+ cells by magnetic separation. CD45+ TIL, pooled from 5 mice, were incubated in the presence and absence of immunodominant tumor peptide, AH-1, at various cell densities. IFNy secreting cells were measured by ELISPOT; peptide specific response was determined by subtracting average of wells without peptide. Each sample was tested in replicates of 6 and is graphed above. Statistical significance was determined with Mann-Whitney non-parametric t test.

An increase in IFNγ secreting cells was observed in MAb 67-treated mice both in the presence and absence of peptide. CD45+ TIL, especially MHC-I-restricted peptide-specific CD8+ cytotoxic T cells, represents activated effector cells following treatment with MAb 67-2. CD45+ TIL then were cultured ex vivo for 48 hours and assayed for cytokine secretion using CBA analysis. Further details of these results were reported in Evans EE, et al., Antibody Blockade of Semaphorin 4D Promotes Immune Infiltration into Tumor and Enhances Response to Other Immunomodulatory Therapies. Cancer Immunol Res. 2015 Jun; 3(6):689-701.

A follow-up study was conducted to examine the effect of MAb 67-2 treatment on the frequency of tumor-specific tumor infiltrating leukocytes (TIL) and secretion of pro-inflammatory cytokines. In this follow-up study, immune cells were isolated from tumors of Colon26 tumor-bearing mice treated in vivo with Control IgG1/MAb2B8 or anti-SEMA4D/MAb67. Total CD45+ TIL were assessed for secreted cytokine levels.

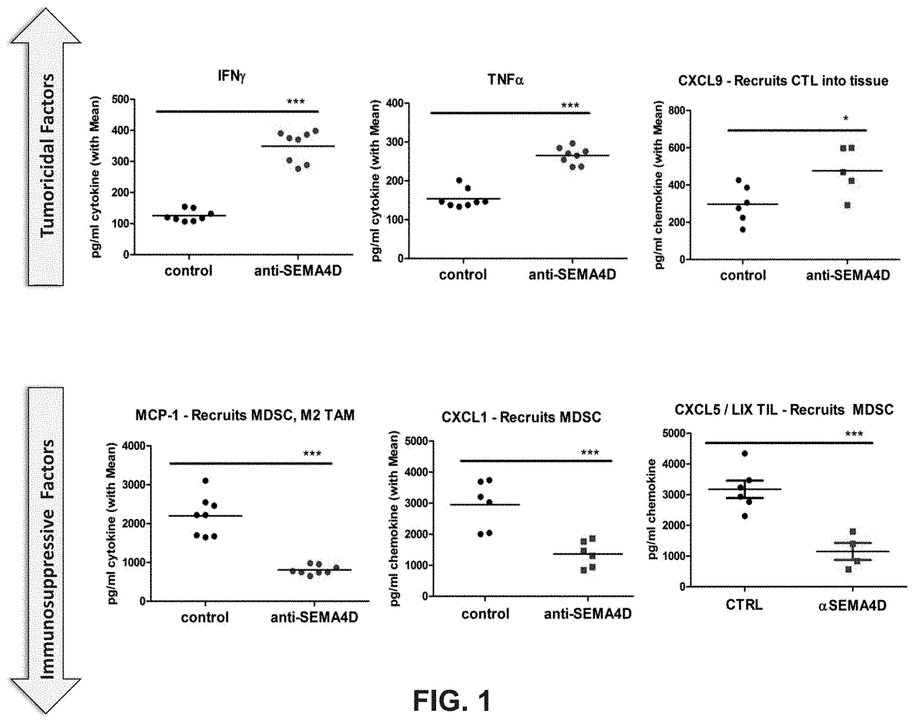

MAb 67-2 treatment increases the secretion of pro-inflammatory factors and reduces secretion of immunosuppressive factors in the tumor microenvironment. As shown in FIG. 1, there is a coordinated shift in the factors secreted by TIL of mice treated with anti-SEMA4D MAb. Increased levels of pro-inflammatory cytokines IFNg and TNFa was observed in the TIL of mice treated with anti-SEMA4D antibody. Additionally, TIL from mice treated with anti-SEMA4D had higher levels of CXCL9, a chemokine that recruits CD8+ T cells into the tumor tissue. In contrast, in these same mice, there was a reduction in immunosuppressive chemokines including CCL2, CXCL1 and CXCL5, which recruit and polarize immunosuppressive cells such as myeloid derived suppressor cells, M2 tumor associated macrophage, and T regulatory cells. Statistical significance was determined with Mann-Whitney non-parametric t test.

### Example 6: Testing the Ability of an Anti-SEMA4D Antibody to Delay Tumor Growth in Mice when Used in Combination with Anti-TGFβ Antibodies

Experimental Design 1. 80,000 MC38 cells were implanted subcutaneously into the flank of C57B1/6J mice (Jackson Labs). Treatment of the following 4 groups, N=15/group, was as follows: (i) control Mouse IgG1/2B8 (10 mg/kg, l×/week for 2 weeks, starting 2 days post tumor injection (dpi)), (ii) anti-SEMA4D/MAb 67-2 (10 mg/kg, l×/week for 2 weeks, starting 2 dpi), (iii) anti-TGFβ/Mab 1D11.16.8 (10 mg/kg, 3×/week for 3 weeks, starting when tumor volume reaches about 130 mm³ (10 dpi)), or (iv) both anti-SEMA4D/MAb 67-2 (initiated 2 dpi, as above) and anti-TGFβ/Mab 1D11.16.8 (as above at 10 dpi).

Experimental Design 2. 500,000 Colon26 cells were implanted subcutaneously into the flank of BALB/cJ mice (Jackson Labs). Treatment of the following 4 groups, N=15/group, was as follows: (i) control Mouse IgG1/2B8 (10 mg/kg, l×/week for 2 weeks, starting 2 days post tumor injection (dpi)), (ii) anti-SEMA4D/MAb 67-2 (10 mg/kg, l×/week for 2 weeks, starting 2 dpi), (iii) anti-TGFβ/Mab 1D11.16.8 (10 mg/kg, 3×/week for 3 weeks, starting when tumor volume reaches about 130 mm³ (10 dpi)), or (iv) both anti-SEMA4D/MAb 67-2 (initiated 2 dpi, as above) and anti-TGFβ/Mab 1D11.16.8 (as above at 10 dpi). Tumor challenge: mice with complete tumor regression (CR) on day 90 were challenged with viable tumor of opposite flank. All mice rejected subsequent tumor challenge, demonstrating immunological memory.

Calculations and Statistics. Tumors were calipered in two dimensions and tumor size was calculated using the formula: Tumor Volume (mm³)=(w2×l )/2, where w=width and l=length, in mm, of the tumor. Tumor weight may be estimated with the assumption that 1 mg is equivalent to 1 mm³ of tumor volume. Mean and median tumor volumes are calculated and graphed for each group at each timepoint. Mean and median calculations are displayed only when >50% animals/group remain on study. Final tumor volume at sac is “carried over” if an animal is sacrificed when >50% animals/group remain on study. Animals sacrificed due to accidental or unexplained death are not included in mean and median calculations, unless tumors reach endpoint volume before death. Two way analysis of variance was applied to the growth curves to determine significance of treatment effect—tumor volume over time. CR refers to the number of complete responders (tumor volume<50 mm³ for>2 consecutive measurements). Differences in regression rates were determined using fisher's exact test, compared to Control.

Time to endpoint for each mouse was calculated with the following equation: TTE=[log 10 (endpoint volume)−b]/m, where TTE is expressed in days, endpoint volume is in mm³, b is the intercept, and m is the slope of the line obtained by linear regression of a log-transformed tumor growth data set. Note: linear regression analysis was also performed using raw tumor volume data. The data set is comprised of the first observation that exceeded the study endpoint volume and the four consecutive observations that immediately preceded the attainment of the endpoint volume. The calculated TTE is usually less than the day on which an animal is euthanized for tumor size. Animals whose tumors do not reach the endpoint are assigned a TTE value equal to the last day of the study. Animals that died from non-treatment-related (NTR) causes were excluded from TTE calculations. Significance of TTE values were determined by applying D'Agostino & Pearson omnibus normality test to TTE values of control and treatment groups. If both groups pass normality test, a parametric t test was applied. Validity of the parametric t test was confirmed if data were normally distributed and variances were significantly different. If variance and/or distribution were ns, a non-parametric test was applied, such as Mann Whitney t-test. Generally, a parametric t test is preferred and expected to be more sensitive in determining significant differences. (a p<0.05 for both tests simply supports the statistical significance of the result).

Tumor growth delay (TGD), is defined as the increase in the median TTE in a treatment group compared to the control group: TGD=T−C, expressed in days, or as a percentage of the median TTE of the control group: % TGD−[(T−C)/C]×100, where T=median TTE for a treatment group, C=median TTE for the control group. Survival was analyzed by the Kaplan-Meier method, employing the logrank test to assess the significance of the difference between the overall survival experiences (survival curves) of two groups, based on their TTE values. The logrank test includes animals that are recorded as NTRm deaths, but excludes all other NTR deaths. The two-tailed statistical analyses were conducted at P=0.05. Since the logrank test is a test of significance and does not provide an estimate of the magnitude of the difference between groups, all levels of significance are reported as either significant or non-significant. Prism reports results as non-significant (ns) at P>0.05, significant (symbolized by “*”) at 0.01<P≤0.05, very significant (“**”) at 0.01<P≤0.01, and extremely significant (“***”) at P≤0.001; **** p<0.0001.

### Design 1 (MC38) Summary:

| | Median | | % | Stat | P- | | % | |
|---|---|---|---|---|---|---|---|---|
| Group | TTE | T-C | TGD | Sig | value | CR | CR | Stat Sig |
| control | 17.68 | — | — | — | — | 1/12 | 8% | — |
| anti SEMA4D | 22.76 | 5.08 | 29% | ns | 0.1999 | 1/10 | 10% | ns |
| anti TGFb + control | 19.38 | 1.69 | 10% | ns | 0.9273 | 0/13 | 0% | ns |
| anti TGFb + anti- | 60.00 | 42.32 | 239% | *** | 0.0003 | 10/15 | 67% | ** vs aTGF |
| SEMA | | | | | | | | and CTRL |
| | | | | | | | | * vs MAb67 |

### Design 2 (Colon26) Summary:

| | Median | | % | Stat | P- | | % | Stat |
|---|---|---|---|---|---|---|---|---|
| Group | TTE | T-C | TGD | Sig | value | CR | CR | Sig |
| Control | 33.54 | — | — | — | — | 2/15 | 13% | — |
| αSEMA4D | 37.05 | 3.51 | 10% | ns | 0.7653 | 1/10 | 10% | ns |
| aTGFb + | 43.54 | 10.00 | 30% | ns | 0.4528 | 2/8 | 25% | ns |
| CTRL | | | | | | | | |
| aTGF + | 75.00 | 41.46 | 124% | ** | 0.0048 | 6/9 | 67% | * |
| aSEMA4D | | | | | | | | |

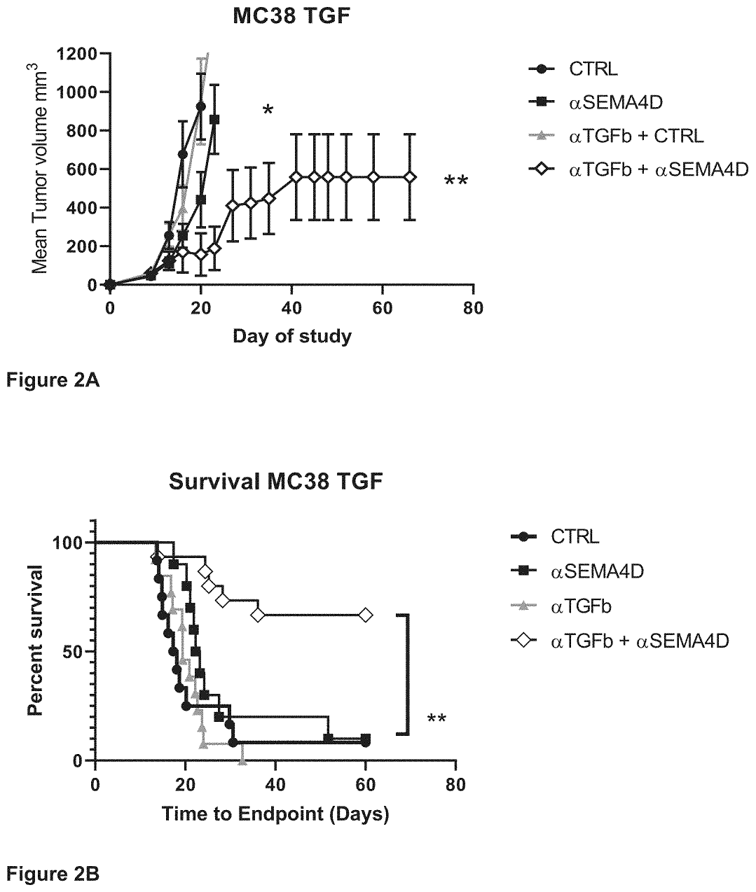

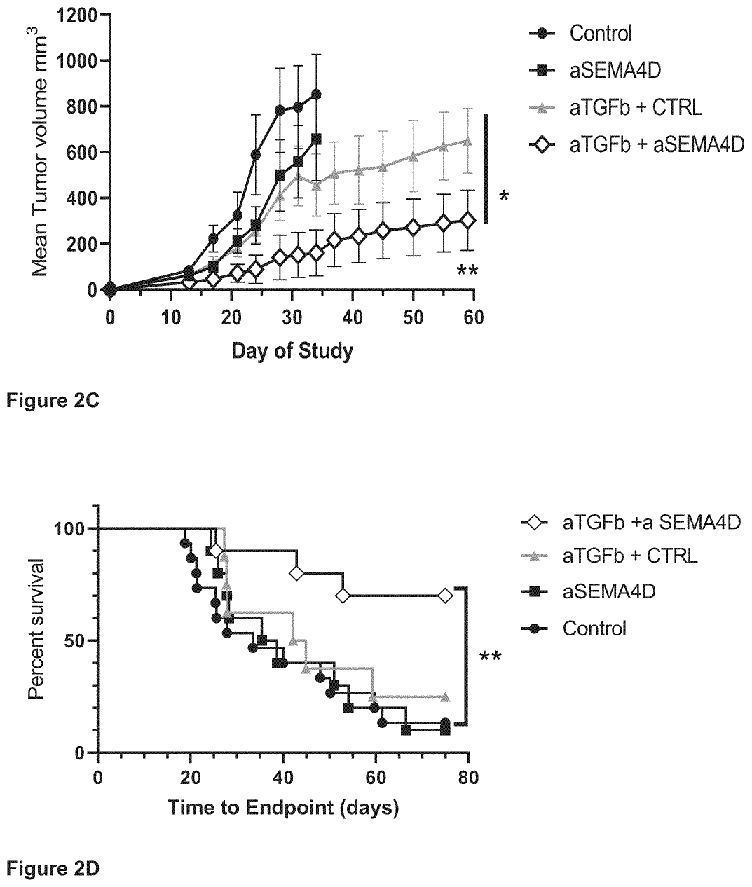

Combination of anti-SEMA4D and anti-TGFβ antibodies delayed tumor growth in mice. Mean tumor volume and Kaplan Meier survival curves, are shown in FIGS. 2A and 2B (Design 1), and 2C and 2D (Design 2), respectively. Combination treatment significantly delays tumor growth and increases the percent of complete tumor regression (CR), compared to control and compared to single agents.

### Example 7: Testing the Ability of an Anti-SEMA4D Antibody to Delay Tumor Growth in Mice when Used in Combination with Anti PD1 Antibodies

Experimental Design. 5×10⁵ Colon26 tumor cells were implanted subcutaneously into the flank of female Balb/c mice. Treatment with control Mouse IgG1/2B8 or anti-SEMA4D/MAb 67-2 was initiated 2 days post inoculation (10 mg/kg, IP, weekly) Each group of mice was also treated with either control rat-Ig or rat anti-PD1/MAbRMP1-14 (10 mg/kg, twice per week, ×2 weeks starting at 9 days post tumor inoculation). There were 15 mice per group. Tumors were measured with calipers 3×/week starting 5 days post implant. Animals were sacrificed when tumor volume reached 1000 mm³.

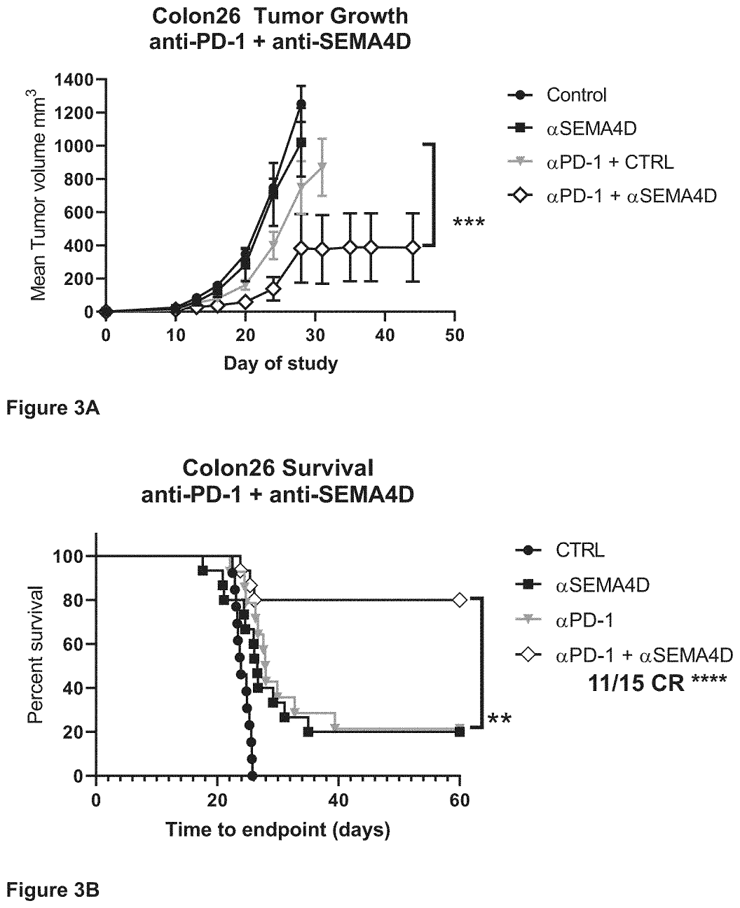

Combination of anti-SEMA4D and anti-PD1 antibodies delayed tumor growth in mice. Tumor growth was measured by calipers and measurements were used to calculate tumor volume using the formula (w²×l)/2, where w=width, smaller measurement, and l=length, in mm, of the tumor. Mean tumor volume and Kaplan Meier survival curves, defined as time to endpoint where tumor volume=1000 mm³, are shown in FIGS. 3A and 3B, respectively. Statistical analysis was conducted using Two-way Analysis of Variance (ANOVA) and Log Rank analysis, respectively, which showed a statistically significant treatment effect with anti-SEMA4D antibody combined with anti-PD1 antibody in Balb/c mice. These finding show that the combination of anti-SEMA4D and anti-PD1 antibodies was more effective than treatment with anti-SEMA4D or with anti-PD1 antibody alone.

The frequency of regressions in Colon 26 tumor model was also measured and is shown in FIGS. 3B. Regression is the lack of palpable tumor, defined by a tumor measuring<50 mm³ for at least two consecutive measurements. Combination of anti-SEMA4D and anti-PD1 antibodies increases the number of regressions in Colon26 tumor model. Regressions for the combination therapy (αSEMA4D+ αPD1 antibodies) are statistically significant compared to Control Ig (p<0.0001) or single agent anti-PD-1 (p=0.0092), as determined by Fisher's Exact test. Further details relating to this Example were reported in Evans EE, et al., Antibody Blockade of Semaphorin 4D Promotes Immune Infiltration into Tumor and Enhances Response to Other Immunomodulatory Therapies. Cancer Immunol Res. 2015 Jun; 3(6):689-701.

### Example 8: Testing the Ability of an Anti-SEAMA4D Antibody to Delay Tumor Growth in Mice when Used in Combination with Anti-TGFb and Anti-PD1 Antibodies

Experimental Design. Treatment of tumor bearing mice with the following groups is as follows: (i) control Mouse IgG1/2B8 (10 mg/kg, l×/week for 2 weeks, starting 2 days post tumor injection (dpi)), (ii) anti-SEMA4D/MAb 67-2 (10 mg/kg, l×/week for 2 weeks), (iii) anti-TGFβ (10 mg/kg, 3×/week for 3 weeks), or (iv) both anti-SEMA4D/MAb 67-2 (as above) and anti-TGFβ (as above). Each group of mice can then also be treated with either control rat-Ig or rat anti-PD1 (100 μg, twice per week, ×2 weeks). Results can be evaluated as described above in Examples 6 and 7.

### Example 9: Testing the Ability of an Anti-SEMA4D Antibody to Delay Growth of in Vivo Cancer Models

Experimental Design. 3×10⁴ Tubo.A5 tumor cells were implanted subcutaneously into the mammary fat pad of female Balb/c mice. Treatment with control Mouse IgG1/2B8.1E7 or anti-SEMA4D/MAb 67-2 was initiated 6 days post inoculation (50 mg/kg, IP, weekly ×6). There were 20 mice per group, however, some mice were excluded from analysis due to premature death before reaching endpoint resulting from ulceration or general ill health. Tumors were measured with calipers 2×/week starting 13 days post implant. Animals were sacrificed when tumor volume reached 800 mm³.

Anti-SEMA4D antibody treatment delayed tumor growth in mice. Tumor growth was measured by calipers and measurements were used to calculate tumor volume using the formula (w²×l)/2, where w=width, smaller measurement, and l=length, in mm, of the tumor. Mean tumor volume and Kaplan Meier survival curves, defined as time to endpoint where tumor volume=800 mm³, are shown in Evans EE, et al., Cancer Immunol Res. 2015 Jun; 3(6):689-701. Statistical analysis was conducted using Two-way Analysis of Variance (ANOVA) and Log Rank analysis, respectively, which showed a statistically significant treatment effect with anti-SEMA4D antibody. The findings show maximal Tumor Growth Delay (133%) with anti-SEMA4D antibody treatment; this is statistically significant compared to using an irrelevant control antibody (p<0.0001), as determined by Mantel Cox Log Rank analysis.

The frequency of tumor regressions in the Tubo.A5 tumor model was also measured and is shown in Evans EE, et al., Cancer Immunol Res. 2015 Jun; 3(6):689-701. Regression is the lack of palpable tumor, defined as a tumor measuring <50 mm³ for at least two consecutive measurements. At 90 days post implant, 85% (12/14) of MAb67-treated mice were tumor-free regressors and one of the 14 had never developed measurable tumor, compared to 0/14 regressions in the mice treated with Control Ig. On day 90, mice who had completely rejected their primary tumors (13/14 of MAb67-treated mice) were challenged with viable Tubo.A5 (30,000) on contralateral side; naive mice were included as controls for graft. All 13 mice that were treated with anti-SEMA4D rejected subsequent tumor challenge, suggesting an immunologic memory response, in contrast to naive mice who did not reject the tumor challenge. The regression frequency is statistically significant compared to Control Ig (p<0.0001), as determined by Fisher's Exact test.

Similar data have been described in a head and neck squamous cell carcinoma (HNSCC) model, as well as in a lung cancer model (LLC). Clavijo PE, et al., Semaphorin4D inhibition improves response to immune checkpoint blockade via attenuation of MDSC recruitment and function. Cancer Immunol Res. 2019 Feb; 7(2):282-291.

### Example 10: Testing the Ability of an Anti-SEMA4D Antibody to Delay Tumor Growth in Mice when Used in Combination with Anti-PD-L1 Antibodies

In general, immuno-oncologists consider anti-PD-1 and anti-PD-L1 to act in a related pathway. This is reflected in parallel approvals for anti-PD-1, Keytruda (pembrolizumab) and Opdivo (nivolumab) as well as anti-PD-L1, Tecentriq (atezolizumab) and Imfinzi (durvalumab) in non-small cell lung cancer (NSCLC) and all of these as well as the anti-PDL1 Bavencio (avelumab) in bladder cancer. Accordingly, an anti-PD-L1 antibody would also be expected to act synergistically with anti-SEMA4D in treating cancer. In fact, a combination of anti-SEMA4D monoclonal antibody Pepinemab in combination with the anti-PD-L1 monoclonal antibody Avelumab is in human clinical trials. Data presented at a recent American Association for Cancer Research meeting, in a poster entitled “Interim results from CLASSICAL-Lung, a phase lb/2 Study of VX15/2503 (pepinemab) in combination with avelumab in advanced NSCLC”. The early results are promising, showing a disease control rate of at least 75% in all subjects.

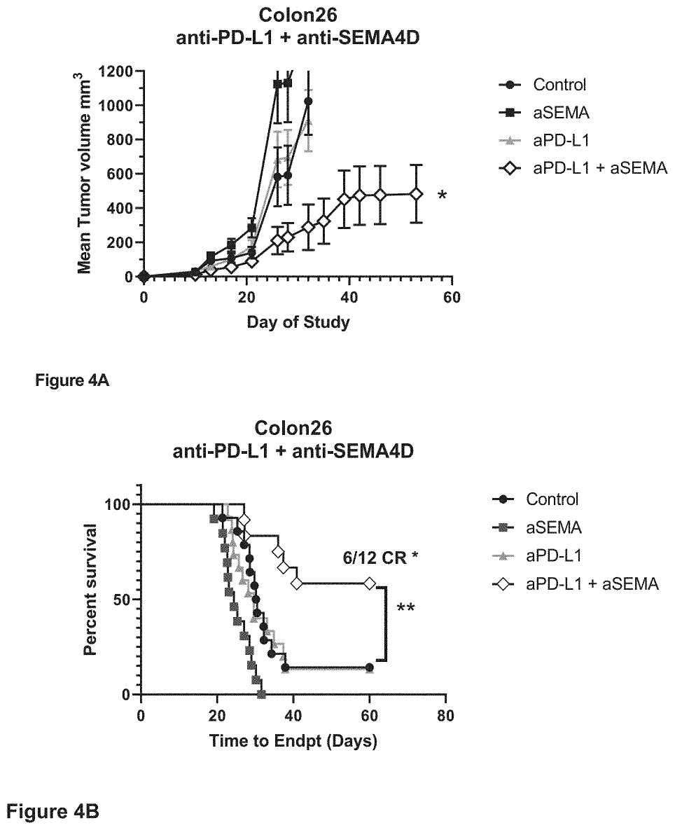

In addition, a synergistic effect between anti-SEMA4D Mab 67 and two other immune checkpoint blockade inhibitors, anti-LAG3 and anti-PD-L1 (the latter shown in FIG. 4). The experiments were done using the same cancer model, Colon26 cells, described above. Specifically, Colon26 (500,000 cells) were subcutaneously implanted into Balb/c mice, that were then treated with anti-SEMA4D Mab 67 (10 mg/kg, weekly IP ×2) and either anti-LAG3/C9B7W (10 mg/kg 2×/week ×4; n=20) or Anti-PD-L1/MAb 10F.9G2 (10 mg/kg 2×/week ×4; n=18). The results for both combination therapies show a highly significant and synergistic reduction in tumor growth and increased survival with the combination therapy.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are also provided.

Amino acids are referred to herein by their commonly known three letter symbols or by the one-letter symbols recommended by the IUPAC-IUB Biochemical Nomenclature Commission. Nucleotides, likewise, are referred to by their commonly accepted single-letter codes.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises or consists of A” is used as a tool to avoid excess page and translation fees and means that in some embodiments the given thing at issue: comprises A or consists of A. For example, the sentence “In some embodiments, the composition comprises or consists of A” is to be interpreted as if written as the following two separate sentences: “In some embodiments, the composition comprises A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the present disclosure, all publications, patents, and patent applications mentioned herein are expressly incorporated by reference therein to the same extent as though each were individually so incorporated.

Having thus described exemplary embodiments of the present disclosure, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A method for inhibiting, delaying, or reducing tumor growth in a subject with cancer, comprising administering to the subject an effective amount of an isolated binding molecule which specifically binds to semaphorin-4D (SEMA4D) and an effective amount of an antibody or antigen-binding fragment thereof that inhibits TGFβ.

2. The method of claim 1, wherein the isolated binding molecule that specifically binds to SEMA4D, and the antibody or antigen-binding fragment thereof that inhibits TGFβ, are administered separately or concurrently.

3. The method of claim 1, wherein administration of the combination of the isolated binding molecule and the antibody or antigen-binding fragment thereof that inhibits TGFβ results in enhanced therapeutic efficacy relative to administration of the isolated binding molecule or the antibody or antigen-binding fragment thereof that inhibits TGFβ alone.

4. The method of claim 1, wherein the antibody or antigen-binding fragment thereof that inhibits TGFβ is a bispecific antibody.

5. The method of claim 1, wherein the binding molecule inhibits SEMA4D interaction with its receptor.

6. The method of claim 7, wherein the receptor is Plexin-B1, Plexin-B2, or CD72.

7. The method of claim 6, wherein the binding molecule inhibits SEMA4D-mediated Plexin-B1, Plexin-Bs, and/or CD72 signal transduction.

8. The method of claim 1, wherein the method further comprises administering to the subject at least one other immune modulating therapy.

9. The method of claim 1, wherein the cancer is selected from the group consisting of carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, and a combination thereof.

10. The method of claim 1, wherein the isolated binding molecule specifically binds to the same SEMA4D epitope as a reference monoclonal antibody VX15/2503 or 67.

11. The method of claim 1, wherein the isolated binding molecule competitively inhibits a reference monoclonal antibody VX18, VX15/2503 or 67 from specifically binding to SEMA4D.

12. The method of claim 1, wherein the isolated binding molecule that specifically binds to SEMA4D comprises an antibody or antigen-binding fragment thereof.

13. The method of claim 12, wherein the antibody or antigen-binding fragment thereof comprises a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively.

14. The method of claim 13, wherein the VH and VL comprise, respectively, SEQ ID NO: 9 and SEQ ID NO: 17 or SEQ ID NO: 10 and SEQ ID NO: 18.

15. The method of claim 8, wherein the immune modulating therapy comprises an immune checkpoint blockade inhibitor.

16. The method of claim 15, wherein the immune checkpoint blockade inhibitor is an anti-PD-1, anti-PD-L1, anti-CTLA4, or an anti-LAG3 antibody.

17. A method for treating a subject having cancer with immunotherapy, comprising:
(a) determining the number of B cells and/or T cells in a subject with cancer; and
(b) administering to the subject an effective amount of an isolated binding molecule which specifically binds to semaphorin-4D (SEMA4D) and an effective amount of an antibody or antigen-binding fragment thereof that inhibits TGFβ, and, optionally, at least one other immune modulating therapy, if the number of B cells and/or T cells in the subject exceeds a predetermined threshold level, thereby treating the subject with cancer.

18. The method of claim 17, wherein the isolated binding molecule that specifically binds to SEMA4D, and the antibody or antigen-binding fragment thereof that inhibits TGFβ, are administered separately or concurrently.

19. The method of claim 17, wherein administration of the combination of the isolated binding molecule and the antibody or antigen-binding fragment thereof that inhibits TGFβ results in enhanced therapeutic efficacy relative to administration of the isolated binding molecule or the antibody or antigen-binding fragment thereof that inhibits TGFβ alone.

20. The method of claim 17, wherein the antibody or antigen-binding fragment thereof that inhibits TGFβ is a bispecific antibody.

21. The method of claim 17, wherein the binding molecule inhibits SEMA4D interaction with its receptor.

22. The method of claim 17, wherein the receptor is Plexin-B1, Plexin-B2, or CD72.

23. The method of claim 22, wherein the binding molecule inhibits SEMA4D-mediated Plexin-B1, Plexin-Bs, and/or CD72 signal transduction.

24. The method of claim 17, wherein the method further comprises administering to the subject at least one other immune modulating therapy.

25. The method of claim 17, wherein the cancer is selected from the group consisting of carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, and a combination thereof.

26. The method of claim 17, wherein the isolated binding molecule specifically binds to the same SEMA4D epitope as a reference monoclonal antibody VX15/2503 or 67.

27. The method of claim 17, wherein the isolated binding molecule competitively inhibits a reference monoclonal antibody VX18, VX15/2503 or 67 from specifically binding to SEMA4D.

28. The method of claim 17, wherein the isolated binding molecule that specifically binds to SEMA4D comprises an antibody or antigen-binding fragment thereof.

29. The method of claim 28, wherein the antibody or antigen-binding fragment thereof comprises a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively.

30. The method of claim 29, wherein the VH and VL comprise, respectively, SEQ ID NO: 9 and SEQ ID NO: 17 or SEQ ID NO: 10 and SEQ ID NO: 18.

31. The method of claim 24, wherein the immune modulating therapy comprises an immune checkpoint blockade inhibitor.

32. The method of claim 31, wherein the immune checkpoint blockade inhibitor is an anti-PD-1, anti-PD-L1, anti-CTLA4, or an anti-LAG3 antibody.

33. A method of treating a subject having cancer with immunotherapy comprising: administering a combination of an effective amount of an isolated binding molecule that specifically binds to semaphorin-4D (SEMA4D) and an effective amount of an antibody or antigen-binding fragment thereof that inhibits TGFβ, to a subject with cancer, wherein administration of the combination results in enhanced therapeutic efficacy relative to administration of the isolated binding molecule or the antibody or antigen-binding fragment thereof that inhibits TGFβ alone.
